(19) United States
(12) Patent Application Publication
(10) Pub. No.: US 2024/0180982 A1
(43) Pub. Date: Jun. 6, 2024

(54) MODIFIED ADENOVIRUS

(71) Applicant: UNIVERSITY COLLEGE CARDIFF CONSULTANTS LIMITED, Cardiff South Glamorgan (GB)

(72) Inventors: Alan PARKER, Cardiff South Glamorgan (GB); James DAVIES, Cardiff South Glamorgan (GB); Emily BATES, Cardiff South Glamorgan (GB)

(21) Appl. No.: 18/284,948

(22) PCT Filed: Mar. 24, 2022
(86) PCT No.: PCT/GB2022/050745

§ 371 Date: Sep. 29, 2023

(30) Foreign Application Priority Data
Mar. 29, 2021 (GB) 2104409.4
Oct. 28, 2021 (GB) 2115496.8
Jan. 7, 2022 (GB) 2200186.1

(51) Int. Cl. A61K 35/761; A61P 35/00; C07K 14/005; C12N 7/00

(52) U.S. Cl. CPC A61K 35/761; A61P 35/00; C07K 14/005; C12N 7/00; C12N 2710/10321; C12N 2710/10322; C12N 2710/10332

(57) ABSTRACT

The invention concerns a modified low seroprevalence adenovirus: a pharmaceutical composition comprising same; and a method of treating cancer using same.

### FIELD OF THE INVENTION

The invention concerns a modified low seroprevalence adenovirus; a pharmaceutical composition comprising same; and a method of treating cancer using same.

### BACKGROUND OF THE INVENTION

Virotherapies exploit the natural ability of a virus to infect, replicate in and lyse cells and direct this ability specifically against tumour cells. The lytic nature of this cell death is highly immunogenic, resulting in enhanced immune recruitment to the tumor microenvironment with corresponding CD8⁺ mediated activation and immune cell mediated cell killing. Certain viruses, such as echoviruses and reoviruses may have natural oncolytic potential, whilst other viruses require genetic manipulation to increase tumour selectivity and enhance cell killing, leading to the development of more effective cancer therapies.

Adenovirus infections are widely distributed across human and animal populations. Infection by human adenovirus (HAdV) can result in a broad array of clinical pathologies, however the majority of infections are asymptomatic or acute and self-limiting.

HAdV are non-enveloped viruses measuring 90-100 nm and are arranged in an icosahedral structure with protruding fiber proteins. The capsid is made up of 240 trimeric hexon proteins, with a pentameric penton base complex located at each vertex. Adenoviral structure has been reviewed extensively. There are 57 canonical HAdV serotypes divided into seven species (A-G) based on serological testing. Over 100 novel adenovirus serotypes have been isolated to date. Primary receptor binding is dependent on the adenoviral serotype but generally species A, C, E and F interact with coxsackievirus and adenovirus receptor (CAR), whilst species B serotypes use CD46 and/or Desmoglein 2 (DSG2). Species D is the largest of the adenovirus species, though many serotypes remain poorly understood. Several Species D serotypes including HAdV-D26 and HAdV-D37 bind sialic acid for cell entry. The extent of this receptor usage within the species is not fully understood.

Adenoviral vectors have important clinical applications ranging from vectors for gene therapy and vaccines to oncolytic viruses. A number of oncolytic HAdV virotherapies have entered clinical trials and have demonstrated safety and feasibility, although delivery and efficacy require optimization before oncolytic adenovirus can be used as an effective cancer therapy.

The most extensively evaluated HAdV, clinically and experimentally, are based on the species C type 5, HAdV-C5. However, there are limitations associated with using HAdV-C5 based vectors. HAdV-C5 binds CAR which is localized at tight junctions between cells and expressed ubiquitously throughout the body. Furthermore, CAR is reported to be downregulated in certain cancers, therefore limiting the utility of CAR as a receptor for active cancer targeting. HAdV-C5 is also known to interact with human coagulation factor X (FX) in serum, via the hexon protein, which mediates transduction to the liver and can lead to hepatotoxicity. High levels of HAdV-C5 pre-existing immunity can also reduce the clinical efficacy of potential HAdV-C5-based oncolytic virotherapies, as a substantial proportion of the population will have experienced an acute adenovirus infection, and many will have developed neutralizing antibodies against common HAdV serotypes, thus rapidly inactivate systemically delivered therapeutic vectors.

Activation of anti-tumor immunity, whilst dampening the innate host anti-viral immune response, is essential to the success of oncolytic adenovirotherapies. Previous work in our laboratory has addressed the above limitations through generation of the HAdV-C5NULL-A20 vector (27). The resulting Ad5NULL vector was retargeted to αvβ6 integrin through insertion of a 20 amino acid peptide, NAVPNLRGDLQVLAQKVART, termed A20, native to foot and mouth disease virus (FMDV). A20 modified viruses selectively infected cells expressing the αvβ6 integrin, a surface protein which is upregulated in several cancer types, including breast, ovarian, pancreatic, and colorectal.

Here, we have modified a D species adenovirus, HAdV-D10. HAdV-D10 is a rare serotype isolated from the eyes of patients with keratoconjunctivitis. Its structure and interaction with host cell receptors is poorly understood. We have optimized this virus to generate a low seroprevalence, highly tumour selective agent for optimal delivery to tumours.

Following generation of the crystal structure of HAdV-D10, we have discovered HAdV-D10 has a fiber knob protein that weakly interacts with both CAR and sialic acid and therefore requires modification to increase its transduction efficacy. It also follows that HAdV-D10 does not function via the typical host cell binding receptors, which may be advantageous given that certain cancers downregulate such typical binding proteins. We have therefore targeted HAdV-D10 to cancer cells by recombinantly expressing αvβ6 integrin using the A20 peptide native to foot and mouth disease virus (FMDV). We then demonstrated improved infectivity and selective killing of αvβ6 integrin positive cancer cell lines. Moreover, we also surprisingly discovered HAdV-D10 does not bind human coagulation factor X (FX), thus avoiding hepatotoxicity and off-site targeting that is common to more typical infectious adenovirus serotypes (such as HAdV-C5). Further still, and notably, the modified virus also evades neutralisation in human serum thus maintaining clinical efficacy.

### STATEMENTS OF THE INVENTION

According to a first aspect of the invention there is provided a modified D species human adenovirus of serotype 10, HAdV-D10, comprising:

a binding epitope with selective affinity for αvβ6 integrin, comprising or consisting of a 20 amino acid peptide, NAVPNLRGDLQVLAQKVART (SEQ ID NO: 1), termed A20 and native to foot and mouth disease virus (termed HAdV-D10.A20); and any one or more of the following features:
a) an IC₅₀ for the coxsackievirus and adenovirus receptor (CAR) greater than 0.001 μg/10⁵ cells; and/or
b) a lack of binding to human coagulation factor X (FX); and/or
c) transduction ability in the presence of serum neutralising antibodies.

The modified HAdV-D10 virus of the invention is advantageous because it does not require multiple modifications, typically required for other serotypes, to prevent off-site adverse targeting; it exhibits remarkable organ specific targeting and experiences no loss of activity in HAdV immune reactive host serum.

As is known to those skilled in the art, in humans, there are 57 accepted human adenovirus serotypes (Ad-1 to 57) classified into seven species (Human adenovirus A to G): A—12, 18, 31; B—3, 7, 11, 14, 16, 21, 34, 35, 50, 55; C—1, 2, 5, 6, 57; D—8, 9, 10, 13, 15, 17, 19, 20, 22, 23, 24, 25, 26, 27, 28, 29, 30, 32, 33, 36, 37, 38, 39, 42, 43, 44, 45, 46, 47, 48, 49, 51, 53, 54, 56; E—4; F—40, 41; and G—52. Accordingly, reference herein to HAdV-D10 refers to Adenovirus serotype 10 belonging to the D-subclass of adenovirus, such as that according to NCBI accession number AB724351.1 and the protein domain sequences set forth therein.

In a preferred embodiment of the invention said A20 peptide is inserted into the DG loop of HAdV-D10.A20 fiber knob protein, preferably at a position between amino acids 304-305 of the fibre knob protein, as set forth in NCBI accession number AB724351.1, specifically accession BAM66698. Unexpectedly, it has been found that when attempting to insert the A20 binding epitope, or targeting peptide, in other locations of the fiber knob protein, such as in the H1 loop (as is typically undertaken for adenovirus serotype 5, Ad5) viral particle formation was prevented. This therefore tends to indicate a preferred insertion loop for the binding epitope. In one embodiment of the invention insertion into said DG loop at position 304-305 involves the use of a short peptide sequence, or linker, such as SKKY. However, as the skilled person will appreciate, other amino acids or peptide linkers may be used with equal effect.

We show herein HAdV-D10.A20 can engage and utilise αvβ6 integrin as a tumour selective cell entry receptor and this function is mediated by the A20 peptide (SEQ ID NO: 1). A20 was originally derived from foot-and-mouth disease virus (FMDV) capsid protein VP1 and has a natively high affinity to αvβ6 integrin. αvβ6 integrin is expressed in a third of ovarian cancers and in a variety of other epithelial cancers and is non-detectable in healthy adult tissues. Therefore, as will be appreciated by those skilled in the art, through expression and incorporation of this sequence in the modified virus, the modified virus can selectively target αvβ6 integrin in cancers such as, but not limited to, lung, breast, esophageal squamous cell carcinoma, ovarian, pancreatic, cervical, head and neck cancer, oral cancer, cancer of the larynx, skin cancer, kidney cancer and colorectal cancer cells. We also show herein no cell killing by HAdV-D10.A20 in cells (e.g. A549 cells) that do not express αvβ6 integrin, thus demonstrating the selectivity of the αvβ6 integrin recombinant binding feature. In this respect, our work has shown a significant increase of HAdV-D.A20 was observed in tumour (p=<0.05) compared to levels in the liver, lung, heart, kidney, and spleen.

In a further preferred embodiment of the invention said modified D species human adenovirus, HAdV-D10.A20, has an IC₅₀ for the coxsackievirus and adenovirus receptor (CAR) greater than 0.002 μg/10⁵ cells, more preferably about 0.003 μg/10⁵ cells, i.e., 0.002985 μg/10⁵ cells, that is to say, HAdV-D10 binds CAR with an apparent 10-fold lower affinity, or a 15-fold lower affinity, or a 16.5-fold lower affinity than HAdV-C5 which possesses a binding partner for coxsackievirus and adenovirus receptor (CAR) that facilitates cell transduction. This means HAdV-D10.A20 will preferentially work through αvβ6 integrin binding.

In a further preferred embodiment of the invention said modified D species human adenovirus, HAdV-D10.A20, lacks key binding residues for FX interactions and so is unable to engage and utilize FX as a means of cell entry nor is it susceptible to eliciting hepatotoxicity. This means HAdV-D10.A20 is a safer therapeutic.

A major obstacle contributing to the reduced efficacy of many adenovirus based therapies is the proportion of patients presenting with pre-existing immunity against such adenoviruses.

Use of an alternative, rarely isolated serotypes, HAdV-D10.A20 provides for therapies that are effective for a broader population but also offers a valuable second line of treatment in the case where patients have developed immunity against HAdV therapies, such as HAdV-C5 based virotherapies, over the course of their disease treatment regimen.

In yet a further preferred embodiment still, said adenovirus is further modified to include a molecule which is a transgene encoding an agent such as, but not limited to, a therapeutic agent. This embodiment therefore concerns the delivery of an agent, intracellularly, to exert a therapeutic action on the targeted cancer cell. Examples of an agent may include an agent that directly stimulates an immune responses, for example GM-CSF, IL-12; an agent to indirectly stimulate the immune system, e.g. an antibody (or fragments of an antibody), an immune checkpoint inhibitor to inhibit a co-repressor such as CTLA-4, PD-L1, PD1, or Lag3; a Bi-specific T cell Engaging (BiTE) antibody construct; a Bi-specific natural killer cell engaging (BIKE) antibody construct; an agent that sensitises tumours to a cell based immunotherapy e.g. encoding CD19; an agent that depletes regulatory T cells within the tumour microenvironments, anti-CD25 antibodies; an agent that sensitises a tumour to radiotherapy or for imaging e.g. by encoding sodium/iodide symporter (NIS) or somatostatin receptor type 2 (SSTR2)). Alternatively, the transgene may encode a therapeutic agent that is directly toxic to a tumour cell, e.g. by encoding the transgene Reduced Expression in Immortalized Cells (REIC/DKK3), or an enzyme that sensitises a cancer cell via conversion of a non-toxic prodrug into a toxic drug e.g. cytosine deaminase, nitroreductase, thymidine kinase. Other transgenes known in the art and useful in the treatment of cancer may be used in the working of the invention.

Accordingly, in a preferred embodiment of the invention said modified adenovirus comprises a molecule encoding at least one transgene as herein described, ideally said molecule is cDNA.

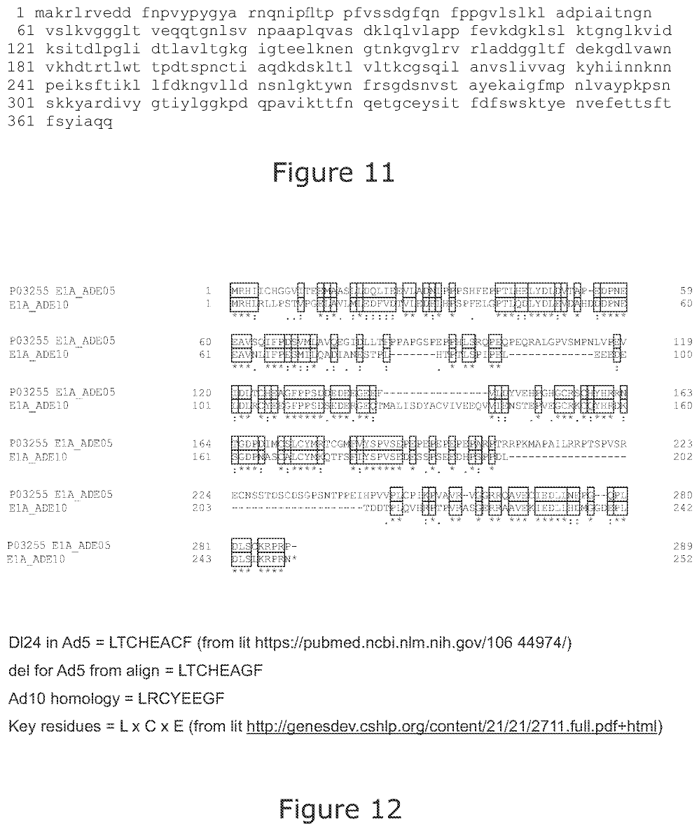

In yet a further preferred embodiment still, said adenovirus is further modified to include an 8 amino acid deletion at position 103-110 (LRCYEEGF; SEQ ID NO: 2) in the E1A gene as set forth in accession number AB724351.1 to restrict viral replication. This deletion is best shown having regard to FIG. 12 wherein the deletion is compared to the corresponding deletion in HAdV-5.

Yet more preferably still, alternatively said adenovirus is further modified to delete one or more of the E1 and/or E3 genes, to render the virus replication deficient. However, as will be appreciated, for the purpose of a cancer lytic virotherapy it is preferred that the virus is replication competent.

In yet a further preferred embodiment still, said adenovirus is further modified to include a 24-base pair deletion dl922-947 (Δ24 mutation) in the E1A gene to restrict viral replication to pRB-defective cells.

Yet more preferably still, said adenovirus is further modified to include a single adenine base addition at position 445 within the endoplasmic reticulum (ER) retention domain in E3/19K (T1 mutation) for enhanced oncolytic potency.

In a yet further preferred embodiment, said adenovirus is further modified to include an adenovirus death protein (ADP). As is known to those skilled in the art, ADP is a type III membrane glycoprotein that ultimately localizes to the nuclear membrane, endoplasmic reticulum and Golgi of an infected cell and is encoded in the E3 transcription unit of several Adenovirus serotypes, including Ad1 (Uniprot Accession number AAQ10560.1), Ad2 (Uniprot Accession number AAA92222.1), Ad5 (Uniprot accession number AP_000221.2), Ad6 (Uniprot accession number ACN88121.1), and Ad57 (Uniprot accession number ADM46163.1). ADP is natively expressed from the E3 promoter at low levels early in infection, when viral proteins affecting cell cycle regulation, inhibition of apoptosis, immune evasion and viral DNA replication are expressed, however, later in the viral replication cycle, when progeny virions are assembled, ADP is expressed at high levels from the major late promoter (MLP) and is thought to contribute to membrane rupture to release assembled virions. Therefore, by incorporating the ADP protein, one improves the oncolytic potential of therapeutic adenovirus, which in combination with cancer cell selectivity, promotes cancer cell death. Preferably, the ADP is selected from the group comprising: Ad1 (Uniprot Accession number AAQ10560.1), Ad2 (Uniprot Accession number AAA92222.1), Ad5 (Uniprot accession number AP_000221.2), Ad6 (Uniprot accession number ACN88121.1), and Ad57 (Uniprot accession number ADM46163.1), and more preferably still that of Ad2 (Uniprot Accession number AAA92222.1) or Ad5 (Uniprot accession number AP_000221.2).

In preferred embodiments, said transgenes and/or ADP is inserted in the E1 and/or E3 region, ideally under the control of native promoters for expression or alternatively using a non-native and strong expressing promoter as known in the art such as, but not limited to, the CMV promoter.

Yet more preferably still, said adenovirus is further modified wherein the E4orf6 region was replaced with the HAdV-C5 E4orf6 to improve viral propagation.

It follows form the above that the modified adenovirus of the invention has been engineered to target cancer cells and stimulate a response against cancer and specifically in a tumour environment.

Accordingly, in a further aspect the invention concerns a pharmaceutical composition comprising the modified adenovirus according to the invention and a pharmaceutically acceptable carrier, adjuvant, diluent, or excipient.

Compounds for use in medicine will generally be provided in a pharmaceutical or veterinary composition and therefore according to a yet fifth aspect of the invention there is provided a pharmaceutical composition comprising the adenovirus as defined herein and a pharmaceutically acceptable carrier, adjuvant, diluent, or excipient.

Suitable pharmaceutical excipients are well known to those of skill in the art. Pharmaceutical compositions may be formulated for administration by any suitable route, for example oral, buccal, nasal, or bronchial (inhaled), transdermal or parenteral and may be prepared by any methods well known in the art of pharmacy.

The composition may be prepared by bringing into association the above defined adenovirus with the carrier. In general, the formulations are prepared by uniformly and intimately bringing into association the adenovirus with liquid carriers or finely divided solid carriers or both, and then if necessary shaping the product. The invention extends to methods for preparing a pharmaceutical composition comprising bringing an adenovirus as defined above in conjunction or association with a pharmaceutically or veterinary acceptable carrier or vehicle.

Accordingly, in yet a further aspect the invention concerns a method of treating cancer in a subject comprising administering to a patient an effective amount of a composition comprising a modified adenovirus according to the invention.

Reference herein to an “effective amount” of the adenovirus or a composition comprising same, is to an amount that is sufficient to achieve a desired biological effect, such as cancer cell death. It is understood that the effective dosage will be dependent upon the age, sex, health, and weight of the recipient, kind of concurrent treatment, if any, frequency of treatment, and the nature of the effect desired. Typically, the effective amount is determined by those administering the treatment.

According to a further aspect of the invention there is provided the modified adenovirus as defined herein for use as a medicament.

According to a yet further aspect of the invention there is provided the modified adenovirus as defined herein for use in the treatment of cancer.

According to still a yet further aspect of the invention there is provided the use of a modified adenovirus as defined herein in the manufacture of a medicament to treat cancer.

It follows from the above that the invention concerns the use of a modified adenovirus—optimized for cancer targeting and surprisingly found to have advantageous features, such as a lack of binding to human coagulation factor X (FX) and/or transduction ability in the presence of serum neutralising antibodies, particularly anti-HAdV-C5 antibodies.

Most preferably the cancer referred to herein includes any one or more of the following cancers: nasopharyngeal cancer, synovial cancer, hepatocellular cancer, renal cancer, cancer of connective tissues, melanoma, lung cancer, bowel cancer, colon cancer, rectal cancer, colorectal cancer, brain cancer, throat cancer, oral cancer, liver cancer, bone cancer, pancreatic cancer, choriocarcinoma, gastrinoma, pheochromocytoma, prolactinoma, T-cell leukemia/lymphoma, neuroma, von Hippel-Lindau disease, Zollinger-Ellison syndrome, adrenal cancer, anal cancer, bile duct cancer, bladder cancer, ureter cancer, brain cancer, oligodendroglioma, neuroblastoma, meningioma, spinal cord tumor, bone cancer, osteochondroma, chondrosarcoma, Ewing's sarcoma, cancer of unknown primary site, carcinoid, carcinoid of gastrointestinal tract, fibrosarcoma, breast cancer, Paget's disease, cervical cancer, colorectal cancer, rectal cancer, esophagus cancer, gall bladder cancer, head cancer, eye cancer, neck cancer, kidney cancer, Wilms' tumor, liver cancer, Kaposi's sarcoma, prostate cancer, lung cancer, testicular cancer, Hodgkin's disease, non-Hodgkin's lymphoma, oral cancer, skin cancer, mesothelioma, multiple myeloma, ovarian cancer, endocrine pancreatic cancer, glucagonoma, pancreatic cancer, parathyroid cancer, penis cancer, pituitary cancer, soft tissue sarcoma, retinoblastoma, small intestine cancer, stomach cancer, thymus cancer, thyroid cancer, trophoblastic cancer, hydatidiform mole, uterine cancer, endometrial cancer, vagina cancer, vulva cancer, acoustic neuroma, mycosis fungoides, insulinoma, carcinoid syndrome, somatostatinoma, gum cancer, heart cancer, lip cancer, meninges cancer, mouth cancer, nerve cancer, palate cancer, parotid gland cancer, peritoneum cancer, pharynx cancer, pleural cancer, salivary gland cancer, tongue cancer and tonsil cancer.

More preferably, said cancer is selected from the group comprising: ovarian cancer, pancreatic cancer, oesophageal cancer, lung cancer, cervical cancer, head and neck cancer, oral cancer, cancer of the larynx, skin cancer, breast cancer, kidney cancer, and colorectal cancer.

According to a further aspect there is provided an adenovirus for use in the treatment of cancer, and a method comprising the use of same, in a subject wherein said subject exhibits pre-existing immunity against an adenovirus such as, but not limited to one or more of: HAdV-C5, HAdV-E4, HAdV-B11, HAdV-D26, HAdV-48, the chimera HAdV-5/3 and Chimp adenovirus. As will be known to those skilled in the art, pre-existing immunity can be determined by numerous means known to those skilled in the art such as detection of host serum neutralising adenoviral antibodies to the adenovirus of interest.

In the claims which follow and in the preceding description of the invention, except where the context requires otherwise due to express language or necessary implication, the word “comprises”, or variations such as “comprises” or “comprising” is used in an inclusive sense i.e. to specify the presence of the stated features but not to preclude the presence or addition of further features in various embodiments of the invention.

All references, including any patent or patent application, cited in this specification are hereby incorporated by reference. No admission is made that any reference constitutes prior art. Further, no admission is made that any of the prior art constitutes part of the common general knowledge in the art.

Preferred features of each aspect of the invention may be as described in connection with any of the other aspects.

Other features of the present invention will become apparent from the following examples. Generally speaking, the invention extends to any novel one, or any novel combination, of the features disclosed in this specification (including the accompanying claims and drawings). Thus, features, integers, characteristics, compounds, or chemical moieties described in conjunction with a particular aspect, embodiment or example of the invention are to be understood to be applicable to any other aspect, embodiment or example described herein, unless incompatible therewith.

Moreover, unless stated otherwise, any feature disclosed herein may be replaced by an alternative feature serving the same or a similar purpose.

Throughout the description and claims of this specification, the singular encompasses the plural unless the context otherwise requires. In particular, where the indefinite article is used, the specification is to be understood as contemplating plurality as well as singularity, unless the context requires otherwise.

An embodiment of the present invention will now be described by way of example only with reference to the following wherein:

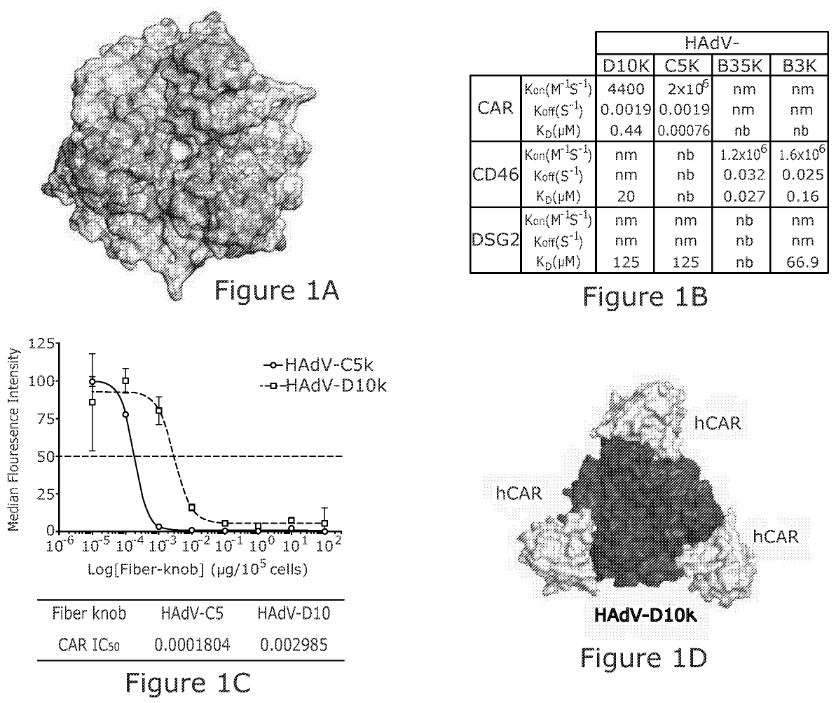

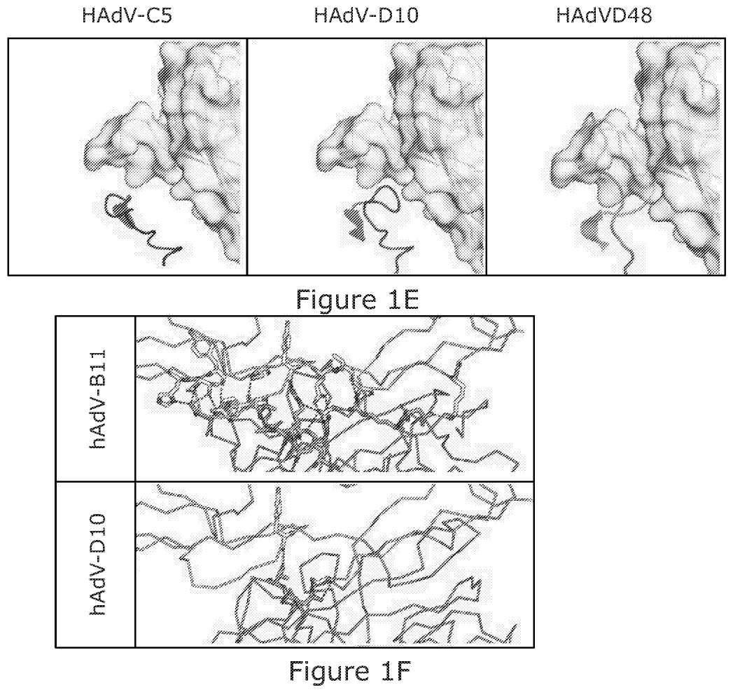

FIG. 1 Shows characterization of the HAdV-D10 fiber knob (HAdV-D10k) and its binding receptors

A) The crystal structure of the HAdV-D10 fiber knob protein. B) Surface plasmon resonance data demonstrates HAdV-D10k binding to CAR, CD46 and DSG2 (nm=kinetics too fast to measure, nb=no binding, Green=specific binding detected). C) Recombinant HAdV-D10k and HAdV-C5k protein binding in CHO-CAR cells using a titration of hCAR specific primary antibody and an Alexa-488 tagged secondary antibody. Data is shown as median fluorescence intensity (MFI) with SD and IC50 values are shown in table. D) Trimeric HAdV-D10 fiber knob (red) shown in complex with hCAR receptor (white). E) Predictive modelling of the hCAR (white) and DG loop interaction of HAdV-D10 (red) in comparison with hAdV-C5 (blue) and HAdV-D48 (green). F) Predictive modelling of CD46 binding sites for HAdV-D10 (cyan) and hAdV-B11 (green), a known CD46 binding adenovirus. Red dashes indicate binding potential. Structural analysis performed using Pymol.

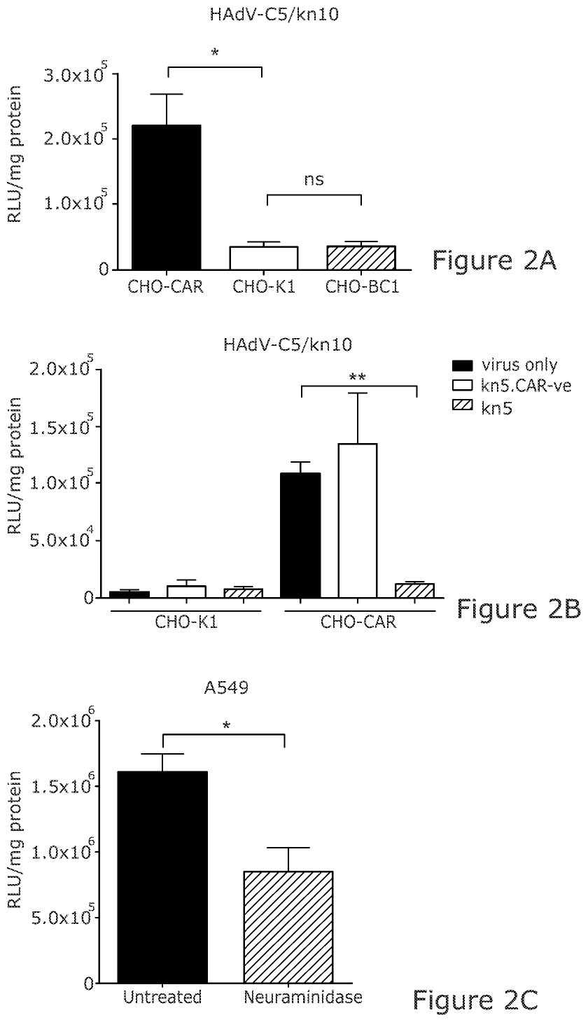

FIG. 2 Shows transduction of HAdV-C5 pseudotype with HAdV-D10 fiber knob

A) Transduction of HAdV-C5/kn10 in cells expressing CAR (CHO-CAR) and CD46 (CHO-BC1). Cells were infected at a viral load of 5000 vp/cell and luciferase production was measured at 48 hours. B) Transduction of both the HAdV-C5/kn10 pseudotype in the presence of hAdV-C5 recombinant knob protein for CAR blocking and HAdV-C5K with a 477YT mutation that ablates CAR binding. C) Neuraminidase assay determines HAdV-C5 pseudotype with HAdV-D10k binding to sialic acid in A549 cells. ns, p>0.05; *, p<0.05; **, p<0.01; ***, p<0.001; ****, p<0.0001.

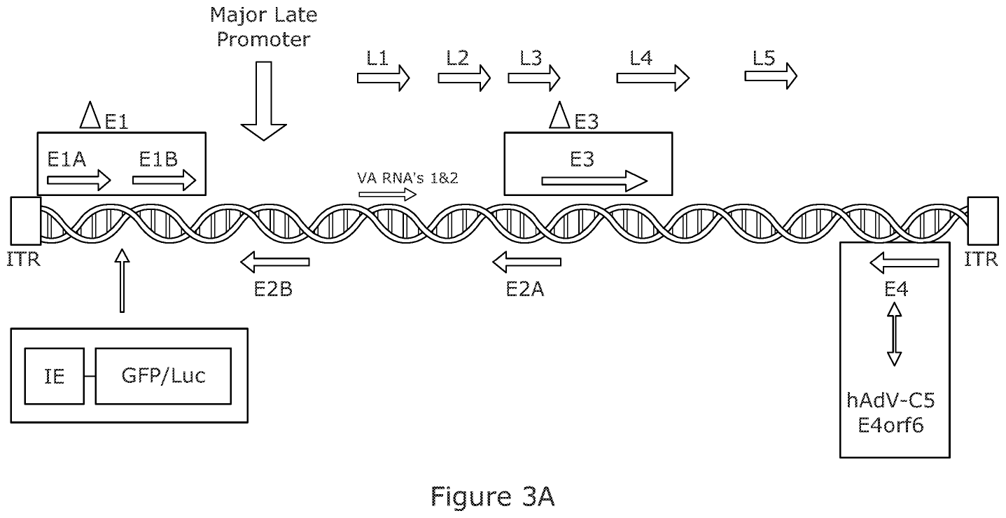

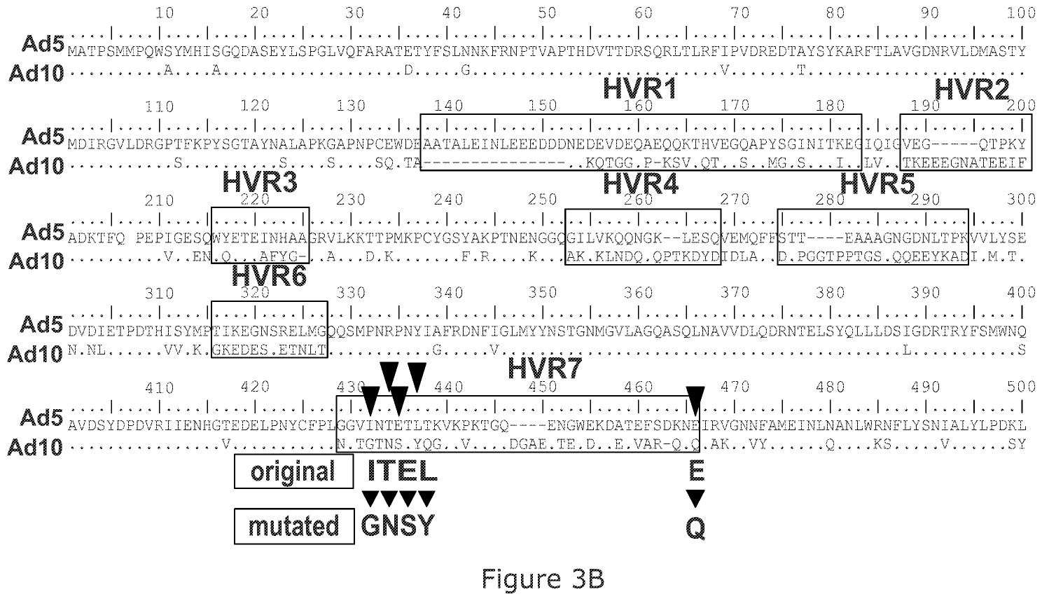

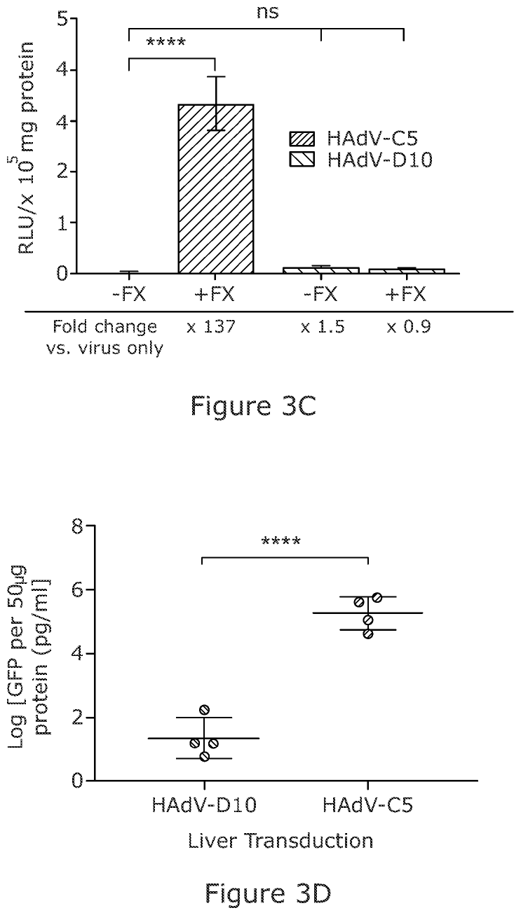

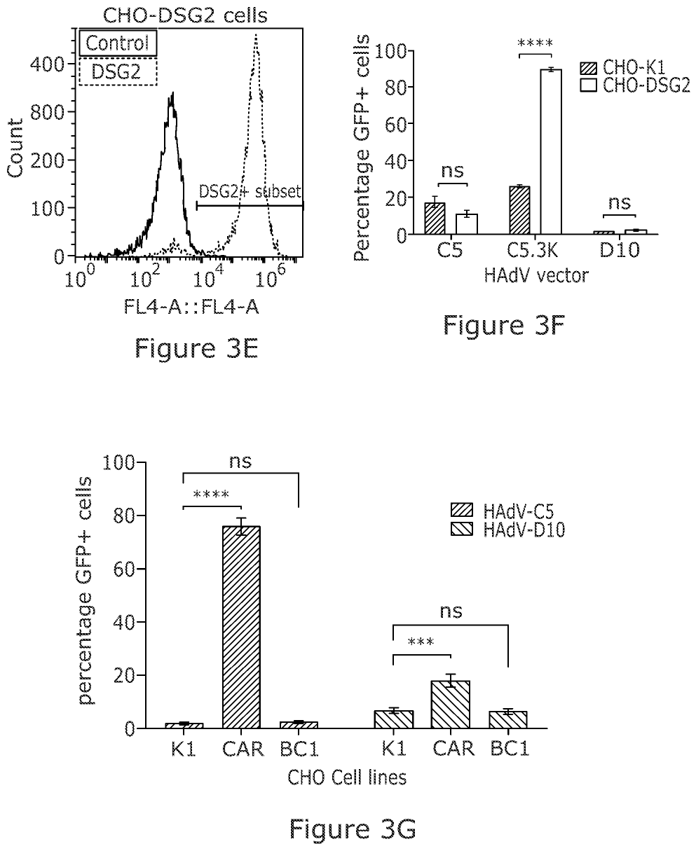

FIG. 3 Shows HAdV-D10 binding to coagulation factor X (FX) and the vector does not use DSG2 or CD46 for cell entry

A) Schematic representing modifications made during production of HAdV-D10 vector. E1 and E3 genes were deleted indicated by the red box, E4orf6 was replaced with HAdV-C5 E4orf6 as highlighted in blue and green represents insertion of the transgenes GFP and Luciferase under the HCMV IE promoter. B) Amino acid sequence alignment of hexon hypervariable regions (HVR) in HAdV-C5 and HAdV-D10 serotypes. Sites for ‘HVR7’ FX-binding mutation in HAdV-C5 shown in purple arrows (53); ‘original’ and ‘mutated’ amino acids involved in the point mutations are shown in bold black letters. C) CHO-K1 cells were transduced with HAdV-C5 and HAdV-D10 vectors at 5000 viral particles/cell for 3 h and luciferase activity measured 48 h later. D) Biodistribution of HAdV-D10 in vivo, 72 hours post intravenous injection. GFP levels were measured in 50 μg of total protein using GFP Simplestep ELISA (Abcam) and calculated from a duplicate mean and concentration was interpolated from a standard curve and transformed using GraphPad software. Log of mean (n=4) and standard deviation of the mean have been shown. Statistical significance was determined using two-tailed unpaired t tests. ns, p>0.05; *, p<0.05; **, p<0.01; ***, p<0.001; ****, p<0.0001. E) Histogram showing proportion of CHO-DSG2 cell line positivity stained for DSG2 receptor use. F) Transduction of CHO-K1 and CHO-DSG2 cells by HAdV-C5 and HAdV-D10 GFP vectors with HAdV-C5.3k used as a positive control for DSG2 receptor use. G) Transduction of CHO-K1, CHO-BC1 and CHO-CAR cells with HadV-C5 and HAdV-D10 with GFP expression measured 72 hours post infection. Data shown as mean of triplicate values. Error bars represent standard deviation of the mean and fold change is relative to ‘virus only’ conditions for each virus. ns, p>0.05; *, p<0.05; **, p<0.01; ***, p<0.001; ****, p<0.0001.

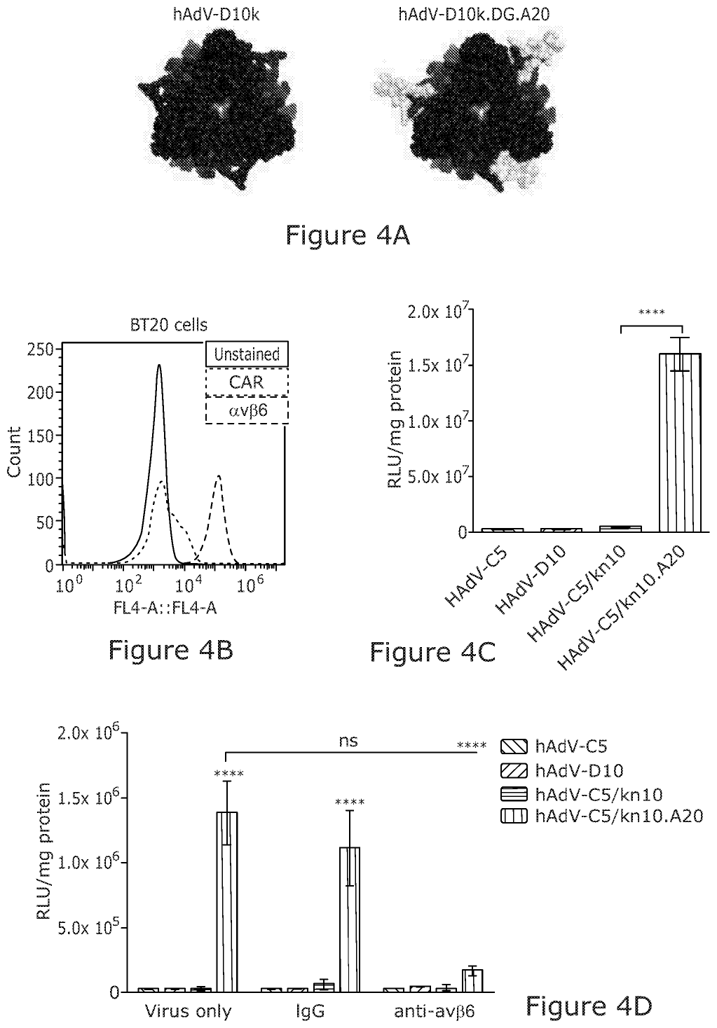

FIG. 4 Shows incorporation of A20 peptide results in αvβ6 targeting

A) Predictive structural modelling of HAdV-D10 knob domains with an A20 targeting peptide insertion in DG structural loop. Structures were based on species D structure Ad19p (PDB ID: 1UXB). A20 amino acid sequence NAVPNLRGDLQVLAQKVART highlighted in green. B) Histogram illustrating proportion of BT20 cells positive for CAR and αvβ6 cell surface receptors determined by flow cytometry. C) BT20 cells transduction of HAdV-C5, HAdV-D10 and HAdV-C5/kn10 pseudotypes. Viral infection was measured by expression of the transgene luciferase 48 hours post infection. D) Transduction of BT20 cells in the presence of receptor blocking antibodies. BT20 cells were preincubated with antibody (IgG and anti-αvβ6) for 30 minutes prior to a 1-hour infection on ice. Unbound virus was removed by washing and luciferase levels were measured 48 hours post infection. Data is plotted as a mean of n=3 with error bars indicating standard deviation. Significance was determined using Two-way ANOVA followed by Tukey's multiple comparison test. ns, p>0.05; *, p<0.05; **, p<0.01; ***, p<0.001; ****, p<0.0001.

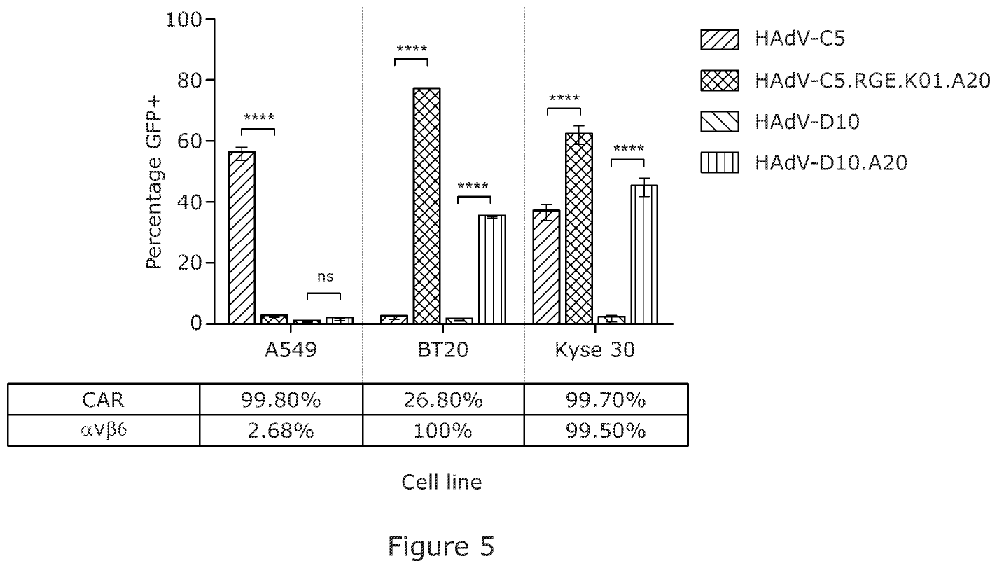

FIG. 5 Shows transduction of HAdV-C5 and HAdV-D10 vectors with the A20 peptide

A549, BT20 and Kyse 30 cells were infected at a viral load of 10 000 vp/cell with both HAdV-C5 and HAdV-D10 vectors and the A20 modified vectors. Expression of the GFP transgene was measured using flow cytometry, 72 hours post infection. The table indicates the percentage expression of the cell surface receptors, CAR and αvβ6, determined by flow cytometry. Data shown is a mean of triplicate values with error bars representing standard error of the mean. Statistical significance was determined by Two-way 702 ANOVA using Tukey's multiple comparisons test. ns, p>0.05; *, p<0.05; **, p<0.01; ***, p<0.001; ****, p<0.0001.

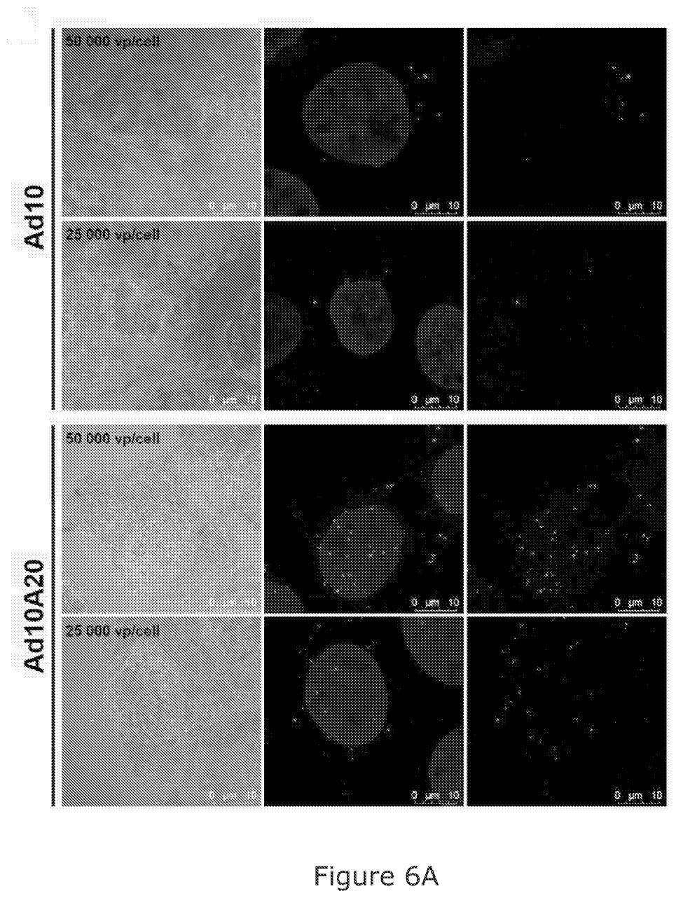

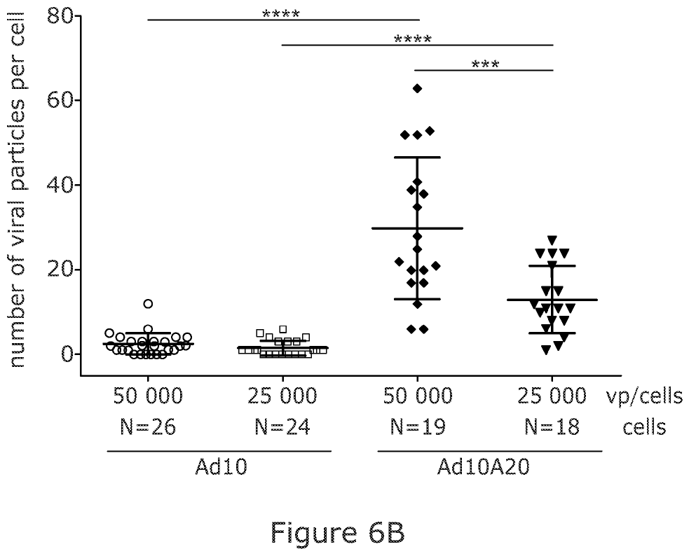

FIG. 6 Microscopy assessing αvβ6 targeting of labelled HAdV-D10 vectors

A) Intracellular trafficking of Alexa Fluor 488-labeled HAdV-D10 and HAdV-D10.A20 in Kyse-30 cells. Green, Alexa Fluor 488-labeled HAdVs; blue, nuclei stained with DAPI; gray, reflection. The images are maximum projections of confocal stacks. Representative confocal images are shown. Scale bars 10 μm. B) Quantification of virus internalization efficiency, expressed as number of viral particles per cell. The horizontal bars represent means, and the error bars indicate standard deviations; the numbers of cells analyzed are indicated (N). *** P<0.001; **** P<0,0001.

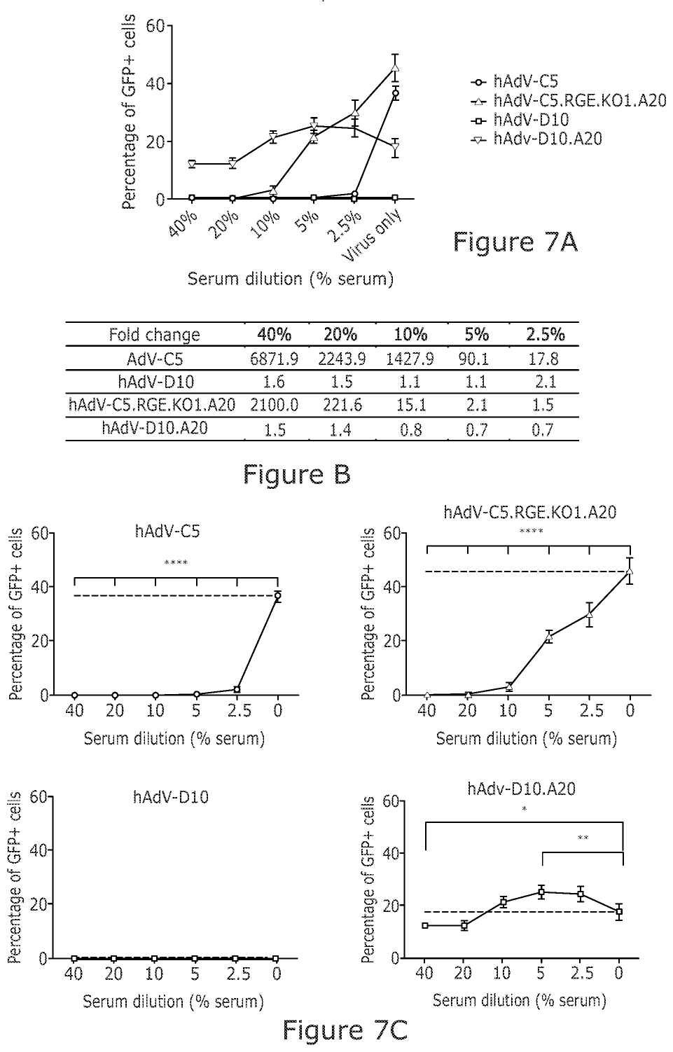

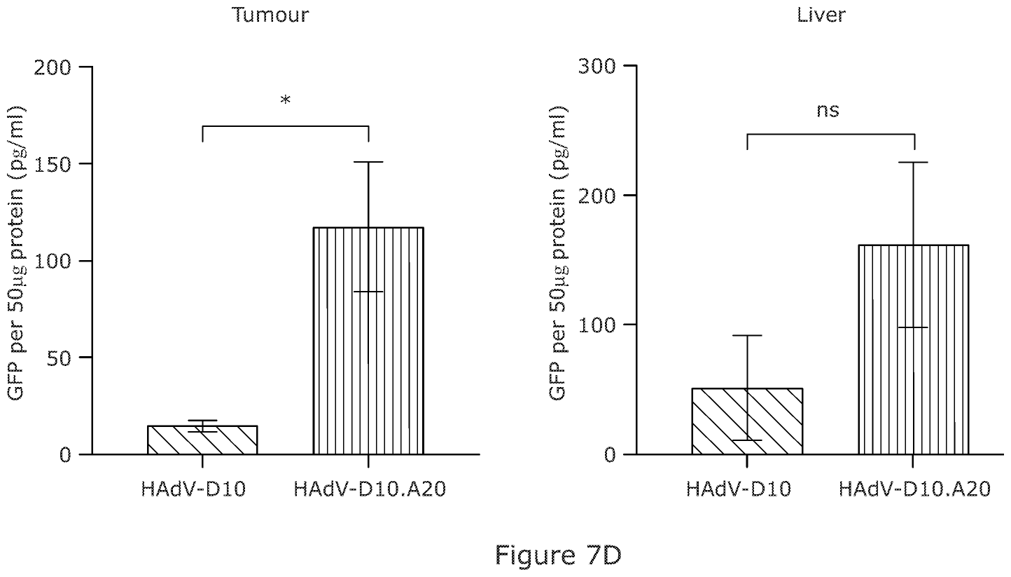

FIG. 7 Shows infection of HAdV-C5 and HAdV-D10 with A20 peptide in the presence of serum

A) Viruses were preincubated for 15 minutes with serum diluted in basal media at different concentrations (40%, 706 20%, 10%, 5%, 2.5% and no serum). Kyse 30 cells were infected with 5000 vp/cell in triplicate for each serum dilution. Expression of the GFP transgene was measured using flow cytometry 72 hours post infection. B) The table indicates fold change when compared to virus infection with no serum for each serum dilution. Data shown is a mean of triplicate values with error bars representing standard deviation of the mean. C) Individual graphs highlighting statistical differences. Statistical significance was determined by a One-way ANOVA using Dunnett's multiple comparisons test. D) GFP ELISA showing biodistribution of HAdV-D10 and HAdV-D10A20 in vivo. Female NSG mice were inoculated subcutaneously with BT20 cells and growth of the xenografts was monitored. GFP expressing HAdV vectors were administered intravenously through injection into the tail vein and tumour and liver were harvested 72 hours post infection. Total protein was extracted and assessed using a BCA assay (Pierce). GFP levels were measured in 50 μg of total protein using GFP Simplestep ELISA (Abcam) and calculated from a duplicate mean and concentration was interpolated from a standard curve using GraphPad software. Data is representative of a mean (n=4) and standard error of the mean. Statistics indicated where significantly different from virus only. ns, p>0.05; *, p<0.05; **, p<0.01; ***, p<0.001; ****, p<0.0001.

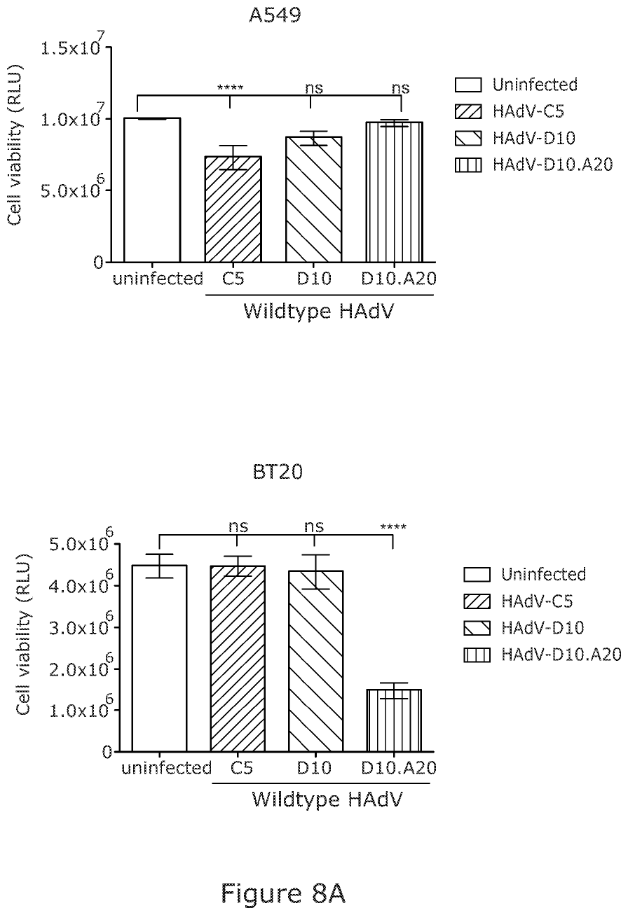

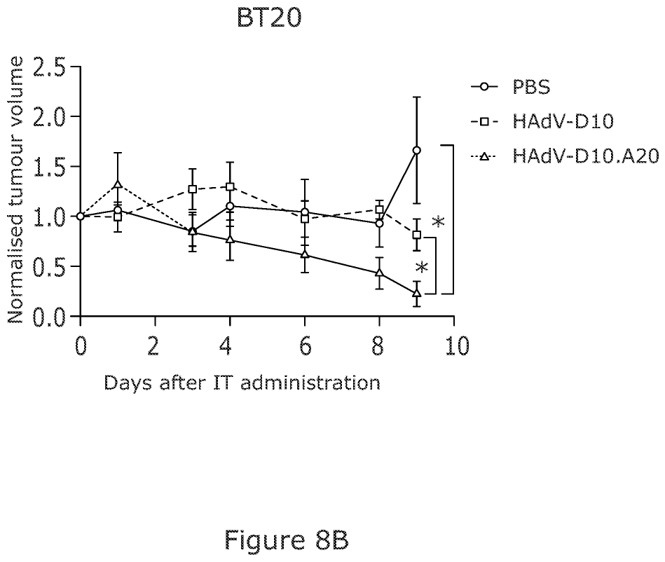

FIG. 8 Shows Cell viability assay with wildtype HAdV-C5, HAdV-D10 and HAdV-D10.A20

A) A549 (αvβ6 low) and BT20 (αvβ6 high) cells were infected at a viral load of 5000 vp/cell with wildtype HAdV-C5 and HAdV-D10 and wildtype HAdV-D10 with the A20 modification. Cell killing was measured hours post infection using CellTiter-Glo® Luminescent Cell Viability Assay (Promega). Data shown is a mean of triplicate values with error bars representing standard deviation. Statistical significance was determined by Two-way ANOVA using Tukey's multiple comparisons test. ns, p>0.05; *, p<0.05; **, p<0.01; ***, p<0.001; ****, p<0.0001. B) HAdV-D10 and HAdV-D10.A20 (10×10\hat{ }10 vp/flank) and PBS controls were administered via intra-tumoural (IT) injection to mice bearing BT20 xenografts. Tumour growth was measured regularly with a caliper for nine days post administration. Data is representative of a mean (n=4) and significance was determined by Mann-Whitney test.

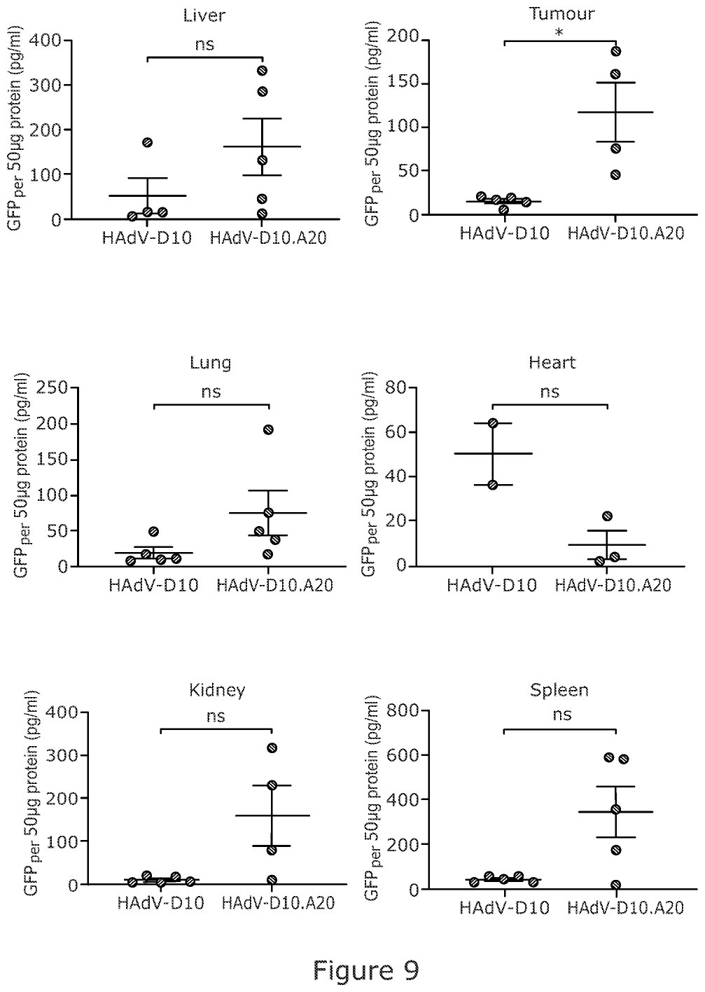

FIG. 9 Shows GFP ELISA showing biodistribution of HAdV-D10 and HAdV-D10A20 in vivo

Biodistribution of HAdV-D10 and HAdV-D10.A20 was assessed by in vivo. Female NSG mice were inoculated sub-cutaneously with BT-20 cells and growth of the xenografts were monitored. GFP expressing HAdV vectors were administered intravenously through injection into the tail vein and organs were harvested 72 hours post infection. Protein was extracted from frozen liver, tumour, lung, kidney, spleen and heart and total protein was assess using a BCA assay (Pierce) GFP levels were measured in 50 μg of total protein using GFP Simplestep ELISA (Abcam) according to manufacturer's instructions. Data is representative of a duplicate mean and concentration was interpolated from a standard curve using GraphPad software. Data that did not fall within the standard curve range has not been plotted. Statistical significance was determined by two-tailed paired t tests. ns, p>0.05; *, p<0.05; **, p<0.01; ***, p<0.001; ****, p<0.0001.

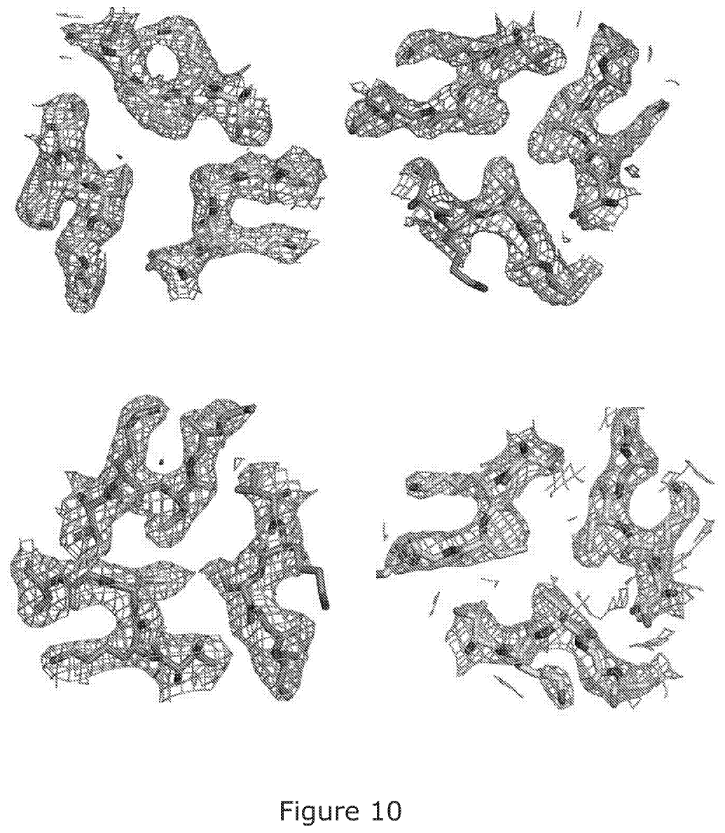

FIG. 10. Electron density around selected parts of the structure. Observed density is blue at 1 Å contour level, positive difference is green at +3 Å, negative difference is red at −3 Å.

FIG. 11. Protein sequence of the Ad10 fiber knob protein, accession number BAM66698.

FIG. 12. Protein sequence showing the E1A (gene) 8 amino acid deletion at position 103-110 (LRCYEEGF) in the E1A protein to restrict viral replication.

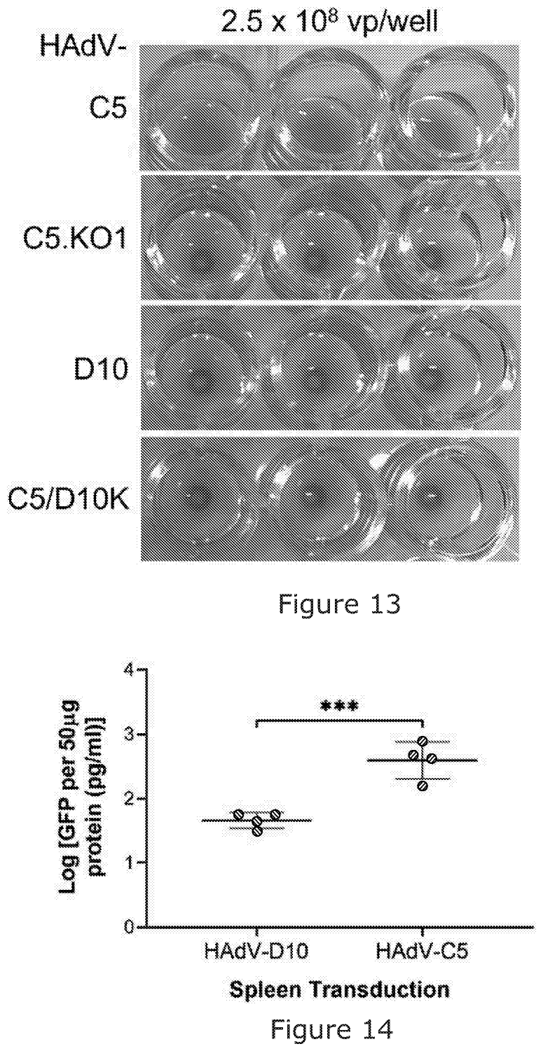

FIG. 13. Haemagglutination assay to demonstrate CAR binding through haemolysis. HAdV-C5, HAdV-C5.KO1, HAdV-D10 and HAdV-C5/D10K were combined with 1% erythrocyte solution in PBS at a concentration of 2.5×108 virus particles.

FIG. 14. Transduction of HAdV-C5 and HAdV-D10 in the spleen, 72 hours post intravenous injection. GFP levels were measured in 50 μg of total protein using GFP Simplestep ELISA (Abcam) and calculated from a duplicate mean and concentration was interpolated from a standard curve and transformed using GraphPad software. Log of mean (n=4) and standard deviation of the mean have been shown. Statistical significance was determined by two-tailed unpaired t tests. ns, p>0.05; *, p<0.05; **, p<0.01; ***, p<0.001; ****, p<0.0001.

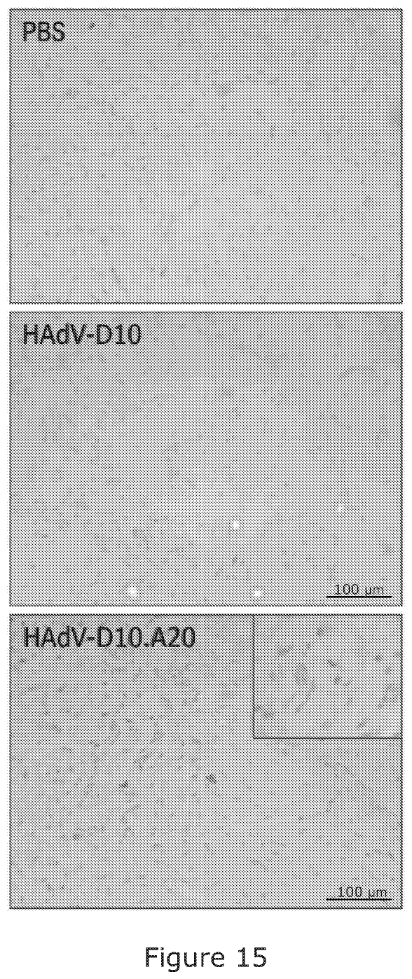

FIG. 15. BT20 tumour sections from mice treated intratumorally with PBS, HAdV-D10 and HAdV-D10.A20 were stained for gamma H2AX as a marker of cell death.

Table 1. HAdv-D10 Fiber Knob Protein Crystal Statistics

Table 2. Primers used in generation of viral vectors

### Materials and Methods

### Generation of Recombinant Fiber Knob Protein

Recombinant HAdV-C5 and HAdV-D10 fiber knob proteins were generated from SG13009 Escherichia coli harbouring pREP-4 plasmid and a pQE-30 expression vector containing the relevant fiber knob encoding DNA sequence. Glycerol stocks were used to inoculate 20 mL LB broth containing 100 μg/mL ampicillin and 50 μg/mL kanamycin and cultured overnight.

The overnight culture was added to 1 L of TB (Terrific Broth, modified, Sigma-Aldrich) supplemented with 100 μg/mL ampicillin, 50 μg/mL kanamycin and 8 mLs of glycerol (0.8%).

Cultures were placed in a shaking incubator (250 rpm) at 37° ° C. until they measured OD600=0.6. Expression of recombinant fiber knob from pQE-30 was induced by addition of IPTG to a final volume of 0.5 mM prior to incubation for 4 hours at 37° C. Bacteria were harvested by centrifugation at 4000 g for 10 minutes at 4° C. Pellets were stored at −80° C. prior to purification. Thawed pellets were resuspended in lysis buffer (50 mM Tris [pH 8.0] 300 mM NaCl, 1% (v/v) NP40, 1 mg/mL Lysozyme, 1 mM ß-mercaptoethanol) and incubated at room temperature for 30 minutes, agitating to aid lysis. Lysates were clarified by a 30-minute centrifugation at 30,000 g and filtered using a 0.22 μm syringe filter (Millipore, Abingdon, UK). Purification was conducted using the AKTA FPLC. Filtered lysates were passed through a 5 mL HisTrap FF nickel affinity chromatography column (GE life sciences, 17525501) at 2.0 mL/min and washed with 5 column volumes into elution buffer A (50 mM Tris [pH 8.0], 300 mM NaCl, 1 mM β-mercaptoethanol). Protein was eluted by 30 min gradient elution from buffer A to B (buffer A+400 mM Imidazole). Fractions were analysed by reducing SDS-PAGE, and fractions containing fiber knob were pooled and further purified using a superdex 200 10/300 size exclusion chromatography column (GE 10/300 GL, GE Life Sciences) in crystallisation buffer (10 mM Tris, [pH 8.0] and 30 mM NaCl). Fractions correlating to area under the chromatogram peaks were collected and analysed by SDS-PAGE. Pure fractions were concentrated by centrifugation in Vivaspin 10,000 MWCO (Sartorius, Goettingen, Germany) proceeding crystallisation.

### Crystallization of HAdV-D10 Fiber Knob (HAdV-D10k) and Structure Determination

Crystallization experiments were set up in a 96-well sitting-drop plate. PACT Premier commercial screen from Molecular Dimensions (UK), was used to equilibrate 200 nL drops of protein against 60 mL drops of screen. The best crystals appeared in the condition 0.2 M CaCl2, 0.1 MES, 20% w/v PEG 6000, pH 6.0. Crystals were harvested into thin plastic loops, cryocooled and transferred to the Diamond Synchrotron Light Source at Harwell, UK. Data collection was conducted at DLS Beamline 104-1. Structure determination of HAdV-D10k was conducted according to previously described methods (48). Reflection data and final model were deposited in the Protein Data Bank (PDB, www.rcsb.org) as entry 6ZC5. A low-resolution form of the structure was also determined and is deposited as entry 6QPM. Full crystallographic refinement statistics and conditions are given in Table 1.

### Surface Plasmon Resonance

A BIAcore 3000TM was used to acquire binding analysis data. CD46, CAR and DSG2 (approximately 500RU) were amine coupled to the surface of a CM5 sensor chip using a slow flow rate of 10 μL/min. Measurements were all performed at a flow rate of 30 μL/min in PBS buffer (Sigma, UK) at 25° C. HAdV-D10 fiber knob protein was purified and concentrated to 178UM. 5×1:3 serial dilutions were prepared for each sample and injected over the relevant sensor chip. The equilibrium binding constant (KD) values were calculated assuming a 1:1 interaction by plotting specific equilibrium-binding responses against protein concentrations followed by non-linear least squares fitting of the Langmuir binding equation. For single cycle kinetic analysis, a top concentration of 200 μM HAdV-D10K was injected, followed by four injections using serial 1:3 dilutions. The KD values were calculated assuming Langmuir binding (AB=B×ABmax/(KD+B)) and the data were analysed using the kinetic titration algorithm (BIAevaluation™ 3.1). Receptor proteins were sourced commercially, as follows:
Recombinant Human Desmoglein-2 Fc Chimera Protein (R&D Systems, 947-DM-100).
Recombinant Human CXADR Fc Chimera Protein (CAR; R&D Systems, 3336-CX-050).
Recombinant Human CD46 Protein (His Tag) (Sino biological, 12239-H08H).

### Predictive Homology Modelling

Fiber-knob proteins were modelled in complex with CAR or CD46 using the template of existing HAdV-C5K (PDB 6HCN) for CAR binding or the HAdV-B11K (PDB 308E) for CD46 binding structures. Non-protein components and hydrogens were deleted from the template model and the fiber knob protein of interest. The Ca chains of each fiber knob proteins were aligned in such a way as to achieve the lowest possible RMSD. Models containing only the HAdV-D10 fiber knob protein and the ligand were saved, and energy minimization was performed, using the YASARA self-parametrising energy minimisation algorithm via the YASARA energy minimisation server. Results were visualised and adapted for publication using PyMoL visualisation software.

### IC50 Assay Using Recombinant Knob Protein

CHO-CAR cells were harvested and 20,000 cells per well were transferred to a 96-well V-bottomed plate (Nunc™; 249662). Cells were washed twice with cold PBS prior to seeding and kept on ice. Serial dilutions of recombinant soluble knob protein were made up in serum-free RPMI-1640 to give a final concentration range of 0.0001-100 μg/10⁵ cells. Recombinant fiber knob protein dilutions were added in triplicate to the cells and incubated on ice for 1 hour. Unbound fiber knob protein was removed by washing twice in cold PBS and primary CAR RmcB (Millipore; 05-644) antibody was added to bind available CAR receptors. Primary antibody was removed after 1 h incubation on ice and cells were washed twice further in PBS and incubated on ice for 30 minutes with Alexa-647 labelled goat anti-mouse F(ab′)2 (ThermoFisher; A-21237). Antibodies were diluted to a concentration of 2 μg/mL in PBS. Cells were washed and fixed using 4% paraformaldehyde and staining detected by flow cytometry on Attune N×T (ThermoFisher). Analysis was performed using FlowJo v10 (FlowJo, LLC) by sequential gating on cell population, singlets and Alexa-647 positive cells. Median fluorescence intensity (MFI) of the Alexa-647 positive single cell population in each sample was determined as previously described and IC50 curves were fitted by non-linear regression using GraphPad software to determine the IC50 concentrations.

### Cell Surface Receptor Staining

Cells were harvested and seeded at a density of 100,000 cells per well in a 96-well V-bottomed plate (Nunc™; 249662). Cells were washed with cold FACs buffer (5% FBS in PBS) before addition of 100 μL primary antibody. Anti-CAR (RmcB, 3022487; Millipore) and anti-αvβ6 (MAB20772; Millipore) were used at a concentration of 2 μg/mL. Primary antibody was removed after 1 h incubation on ice and cells were washed twice in FACs buffer and incubated on ice for 30 minutes with 1:500 dilution of Alexa-647 labelled goat anti-mouse F(ab′)2 (ThermoFisher; A-21237). Stained cells were fixed using 4% paraformaldehyde prior to measurement by flow cytometry on Accuri C6(BD Biosciences). Analysis was performed using FlowJo v10 (FlowJo, LLC) by sequential gating on cell population, singlets and Alexa-647 positive cells.

### Cell Culture

Cell lines were sourced either from American Type Culture Collection (ATCC) or collaborators. Cells were grown at 37° C. in a humidified atmosphere with 5% CO₂ in a Human Tissue Act (HTA) certified cell culture incubator (HERA Cell, Thermo Scientific). Mammalian cell lines were sub-cultured as required (80-100% confluency) in cell line-specific media and supplements. Kyse-30 and A549 were maintained in Roswell Park Memorial Institute 1640 (RPMI; Sigma, #R0883). BT20 cells were grown in Minimum Essential Medium Eagle (EMEM, a modification; Gibco, #11095080). CHO derived cells were cultured in Dulbecco's Modified Eagle Medium: Nutrient Mixture F-12 (DMEM/F-12; Gibco, #10565018). Basal media was supplemented with 10% Foetal Bovine Serum, heat inactivated (FBS; Gibco, #10500-064), 1% L-Glutamine (stock 200 mM; Gibco, #25030-024), 2% penicillin and streptomycin (Gibco, #15070-063).

### Generation of Viral Vectors

Pseudotyped HAdV-C5/kn10 and HAdV-C5/kn10.A20 vectors were produced by AdZ recombineering using previously described methods (33, 49). To generate BAC DNA containing the HAdV-D10 genome HAdV-D10 virus was obtained from ATCC and passaged in A549 cells. Viral stocks were prepared by standard methods and DNA extracted using QIAamp MinElute Virus Spin Kit. A capture BAC containing 500 b.p. homology to each end of the Ad10 genome was generated and used to capture the genome by recombination in SW102 bacteria. This enabled rapid and efficient manipulation of the viral genome by further recombineering. The E1 and E3 genes were deleted to render the vector non-replicative and HAdV-D10 E4orf6 region was replaced with the HAdV-C5 E4orf6 to enhance production in 293 cells. GFP or Luciferase transgenes were inserted under the control of a CMV promoter replacing the E1 region. HAdV-D10 was retargeted to αvβ6 insertion of the A20 peptide into the DG loop of the HAdV-D10 fiber knob. The primers used are detailed in Table 2.

### Virus Preparation and Purification

DNA was amplified using a maxiprep kit as described in the manufacturer's instructions (Nucleobond BAC 100, Macherey-Nagel). DNA concentration was determined using a Nanodrop ND-1000 (Thermo Scientific, UK). Virus particles were generated by lipofectamine transfection onto a T25 CELLBIND flask of T-REX cells or 29386 cells. Cells were collected when CPE was apparent, and virus was amplified using the respective cell lines. Caesium chloride (CsCl) two-step purification method was used to extract pure virus. Alexa-Fluor 488 labelled viruses were prepared using CsCl purification with dialysis into PBS. Viral particles were then incubated for two hours at RT with 20-fold excess of Alexa-Fluor488-TFP (Molecular Probes). Zeba Spin desalting columns (Pierce) were used to purify labelled viral particles. Viral titer was determined using both microBCA and NanoSight (Malvern Panalytical) technology. Viruses were maintained at −80° C. for long term storage.

### Viral Transduction Assays

Cells were seeded at the appropriate density on a sterile 96-microwell Nunc tissue culture plate (Thermo Scientific, #163320) 24 hours prior to infection. Cells were washed with PBS and viruses diluted to the stated concentration in serum free media were added in triplicate. Plates were incubated for 3 hours at 37° C. then virus dilution was replaced with complete growth media. Viral transduction was measured at either 48 or 72 hours post infection. Luciferase expression was detected using Luciferase Assay System kit, according to the manufacturers protocol (#E1501; Promega UK Ltd, Southampton, UK). Protein concentration (mg/mL) determined using a Pierce™ BCA Protein Assay Kit (#23227; Thermo Scientific, Loughborough, UK) and absorbance was measured at A570 nm on an iMark™ Microplate Absorbance Reader (BioRad, Hertfordshire, UK). GFP expression was measured by flow cytometry using Attune N×T (ThermoFisher). Cells were trypsinised, resuspended in FACs buffer (5% FBS in PBS) and transferred to a 96-well V-bottomed plate (Nunc™; 249662).

Cells were washed with cold PBS and fixed using 4% paraformaldehyde for 10 minutes at 4° C. GFP was detected in the BL-1 channel and raw data was analysed using FlowJo v10 (FlowJo, LLC) by gating sequentially on cell population, singlets and GFP positive cells. GFP expression was quantified by percentage of cells positive for GFP compared to an uninfected control.

### Neuraminidase Assay

A549 cells were seeded at a density of 20,000 cells per well and allowed to adhere overnight. Cells were washed twice with PBS before addition of 50 μL of neuraminidase enzyme from Vibrio Choleraa (11080725001, Roche) used at 50 mU/mL. Cells were incubated at 37° C. for 1 hour prior to washing with cold PBS. Viral transduction was carried out as previously described on ice to ensure cleaved sialic acid is not replenished.

### FX Transduction

For assessment of the impact of physiological concentrations of human coagulation FX on transduction efficiency, viral transduction was performed as described for the luciferase assay above. Virus dilutions were prepared in serum-free medium that was supplemented with 10 μg/mL of FX (#HCX0050, Haematologic Technologies, Cambridge Bioscience, Cambridge, UK) for 3 h.

### Confocal Microscopy

Kyse-30 cells were seeded on a coverslip in 24-well plates at a density of 20000 cells per well. The following day cells were infected with Alexa-Fluor 488 labelled viruses at a concentration of 25000 or 50000 vp/cell and transferred to 37° C. for 3 hours. Virus was removed and plates returned to 37° C. for 72 hours after which cells were fixed with 4% paraformaldehyde in PBS for 10 minutes at RT. Coverslips were mounted onto slides using a single drop of VECTASHIELD Antifade Mounting Media (H-100-10) containing DAPI to stain nuclei. Confocal microscopy was carried out using a Leica TC SP2 AOBS scanning microscope and images processed using the Leica Application Suite X (LASX).

### Viral Transduction in Presence of Serum

To determine the effect of neutralising antibodies on viral transduction, serum was collected from the blood of a healthy donor. Serum was serially diluted by half in basal media from 80% to 5%. Serum dilutions were added at a 1:1 ratio with basal media containing 5000 vp/cell giving a final well serum concentration range of 40% to 2.5%. GFP expression was measured by flow cytometry as described.

### Cell Viability Assay

Cells were seeded at a density of 10,000 cells per well in triplicate in a white, opaque bottom 96 well plate (Corning™ 3915). Cells were seeded 24 hours prior to viral infection. Wildtype HAdV-C5, HAdV-D10 and HAdV-D10.A20 were added to cells at a concentration of 5000 vp/cell. The plates were incubated at 37° C. and the viability was measured using CellTiter-Glo® Luminescent Cell Viability Assay (Promega). CellTiter-Glo reagents were prepared according to the manual and 50 μL of the reagent was added to the cells. The plates were protected from light and shaken to fully lyse cells before luminescence was read using a multimode plate reader (FLUOstar Omega, BMG Labtech, Aylesbury, UK).

### In Vivo Studies

Female NSG mice were sub-cutaneously implanted with 6×10\hat{ }6 BT-20 cells/flank and xenograft growth was monitored. When tumours were established, a total of 1×10\hat{ }11 replication deficient virus particles was administered through intravenous tail vein injection to each mouse (n=5). Liver, lung, kidneys, spleen, heart, and tumour were harvested 72 hours post infection. Tissue was stored at −80 degrees. Tissue was homogenized using the TissueRuptor (Qiagen). Protein was extracted from frozen tissue as recommended for the GFP SimpleSTEP ELISA (Abcam, ab171581) and GFP was measured according to the kit protocol. Total protein was determined using a BCA assay (Pierce) and all samples were diluted to 50 μg prior to the ELISA. Absorbance was measured at OD450 using a Cytation 5 microplate reader (Biotek). For replication competent wildtype analysis, 10 days after injection of the cells, HAdV-D10 and HAdV-D10.A20 (10×10\hat{ }10 vp/flank) and PBS controls were administered via intra-tumoural (IT) injection. Tumour growth was measured regularly with a caliper ensuring no more than 15% weight loss was observed. Mice were harvested nine days post administration. For All animal experiments were approved by the Animal Ethics Committee, Cardiff University.

### Statistics

Analysis of raw data was performed using GraphPad unless stated otherwise. Data is shown as a mean of triplicates with standard error (SEM) or standard deviation of the mean (SD). Statistical analysis was carried out as indicated and statistical significance is shown as follows; ns=p>0.05; *=p<0.05; ** 287=p<0.01; ***=p<0.001; ****=p<0.0001.

### Results

HAdV-D10 Knob Binds with Weak Affinity to Known Adenoviral Receptors

We determined the crystal structure of HAdV-D10 fiber knob (HAdV-D10k; FIG. 1A) in two forms, at 2.5 Å and 3.4 Å, deposited in the Protein Data Bank, PDB, as entries 6ZC5 and 6QPM respectively. Crystallization conditions, data collection and refinement statistics are included in Table 1. Electron density maps are shown around selected parts of the structure in FIG. 10. The interactions of species D adenoviruses with cellular receptors are poorly understood. We therefore investigated the binding of HAdV-D10 to several known adenoviral receptors. The binding of HAdV-D10k to three well described adenoviral receptors, CAR (primary receptor for Species C) CD46 and DSG2 (associated with Species B adenovirus interactions), was quantified using surface plasmon resonance (FIG. 1B). HAdV-D10k was able to bind to all three receptors, but the interaction was weak for both CD46 and DSG2 (KD: 20 UM and 125 UM). The on/off rate for these receptors could not be measured, as the receptor-knob complex dissociated too quickly to measure the kinetics. Binding kinetics for HAdV-C5k, HAdV-B35k and HAdV-B3k are also shown, which are controls binding to CAR, CD46 and DSG2 respectively. We demonstrated that HAdV-D10 can form a stronger interaction with CAR than CD46 and DSG2 (0.44 UM). This is still considered a weak interaction in comparison with CAR binding to HAdV-C5 knob (0.76 nM). We investigated the ability of HAdV-D10 to interact with CAR in further detail. IC50 levels of recombinant HAdV-D10 knob proteins were gauged using CHO-CAR cells (FIG. 1C). The binding affinity was quantified using an anti-CAR antibody, followed by staining with a fluorescently labelled secondary antibody. The level of fluorescence was measured using flow cytometry and plotted in GraphPad as median fluorescence intensity (MFI). The data demonstrate that HAdV-D10 binds CAR with an apparent 16.5-fold lower affinity than HAdV-C5 as indicated by the IC50 values. Predictive homology modelling of the trimeric HAdV-D10 knob in complex with CAR confirmed that HAdV-D10k is able to form a structural interface with CAR (FIG. 1D), however the extended DG loop inhibits binding to CAR resulting in an overall lower affinity than HAdV-C5 due to the potential for steric hindrance (FIG. 1E).

We investigated whether the predicted weak interaction between HAdV-D10k and CD46 was sufficient to result in cell attachment and infection. We firstly modelled the interaction between CD46 and HAdV-D10k using predictive homology modelling (FIG. 1F). HAdV-B11 was used as a comparison as a known CD46 binding adenovirus. HAdV-D10 showed far fewer potential binding sites, indicated by the red dashes, than HAdV-B11. Therefore, our homology model confirms the surface plasmon resonance data and that any potential interaction formed with CD46 would be weak.

We next assessed the use of these receptors in viral transduction assays. CHO-K1 cells (expressing no known HAdV receptor), CHO-CAR and CHO-BC1 cells (expressing CAR and CD46 respectively) were infected with the pseudotyped HAdV-C5/kn10 vector (FIG. 2A). HAdV-C5/kn10 was not able to transduce CHO-K1 cells but was able to infect CHO-CAR due to CAR receptor usage. HAdV-C5/kn10 was unable to transduce CHO-BC1, again highlighting a redundancy of CD46 usage by HAdV-D10. This agrees with both our SPR and modelling data and suggests that although weak binding of CD46 was observed, CD46 engagement is not robust enough to result in productive infection. Therefore HAdV-D10 is unable to use CD46 as a mechanism for cell attachment or entry.

After establishing HAdV-D10 is able to bind and use CAR as an entry receptor, we investigated whether HAdV-C5 expressing the HAdV-D10 fiber knob (HAdV-C5/kn10 pseudotype) could use CAR to infect cells (FIG. 2B). CHO-K1 cells and CHO-CAR cells were infected with HAdV-C5/kn10 in the presence of CAR-blocking with either HAdV-C5 recombinant knob (kn5) or HAdV-C5 recombinant knob with a Y477T CAR-binding mutation (kn5.CAR-ve). CHO-K1 cells showed limited viral transduction due to the lack of available receptors for attachment. HAdV-C5/kn10 was able to transduce CHO-CAR cells both without blocking and in the presence of kn5.CAR-ve protein, but transduction was significantly reduced when CAR was blocked using kn5 protein confirming that HAdV-C5/kn10 is both able to bind and use CAR. Additionally, we performed a hemagglutination assay to assess binding of CAR on erythrocytes stimulating haemolysis. HAdV-C5 was used as a positive control for haemolysis and HAdV-C5.KO1 with ablated CAR binding was used as a negative control. As predicted HAdV-D10 and HAdV-C5/D10K demonstrated minimal haemolysis due to weak CAR interactions (FIG. 13).

As HAdV-D10 formed weak interactions with CAR, CD46 and DSG2 it is unlikely they are used as a primary receptor. Several species D adenoviruses have been reported as binding and using sialic acid. To investigate whether HAdV-D10k utilizes sialic acid, we infected neuraminidase-treated A549 cells with HAdV-C5/kn10 (FIG. 2C). Cells treated with neuraminidase showed significant decrease in viral infection (p=<0.05). Although this experiment is not definitive, it does suggest HAdV-C5/kn10 may be able to use sialic acid as an entry receptor. Further structural analysis is required to confirm whether HAdV-D10 is capable of binding sialic acid.

HAdV-D10 does not Interact with Coagulation Factor X (FX)

To generate a HAdV-D10 vector, genomic DNA was captured within a BAC to enable rapid and efficient manipulation of the viral genome (FIG. 3A). The E1 and E3 genes were deleted by recombineering to render the vector non-replicative and the E4orf6 region was replaced with the HAdV-C5 version to enhance production in 293 cells. GFP or luciferase markers were inserted under control of HCMV IE promoter. Coagulation factor X (FX) is known to interact with HAdV-C5 and mediate transduction to the liver resulting in hepatotoxicity and reduced therapeutic effects of HAdV-C5 based virotherapies. Alba et al. (50) identified the FX binding region of HAdV-C5 hexon and key amino acids involved in this interaction through comparison with the hexon HVR7 region of HAdV-D26, known not to bind FX. Modifications to ablate this interaction are required to develop HAdV-C5 based therapies that do not interact with FX. To establish whether HAdV-D10 is capable of binding FX, we performed an alignment of HAdV-C5 and HAdV-D10 hexon hypervariable regions (HVR) highlighting key amino acids involved in FX binding (FIG. 3B). This alignment demonstrates that HAdV-D10 possesses amino acids in the HVR7 region homologous to the mutations described to ablate FX-binding. We therefore predicted that HAdV-D10 was unable to interact with FX based on the amino acid sequence and confirmed this using a viral transduction assay. CHO-K1 cells were infected with either HAdV-C5 or HAdV-D10 in the presence or absence of FX (FIG. 3C) and compared to a virus only control. There was a 137-fold increase in production of the transgene luciferase for HAdV-C5 (p=<0.0001) in the presence of FX, however, this effect was not observed in HAdV-D10 infection where there was no significant difference in infection in presence of FX (0.8-fold change). This was investigated further, in vivo, through biodistribution of HAdV-C5 and HAdV-D10 GFP expressing vectors (FIG. 3D). GFP levels observed in the liver in mice were significantly higher in mice treated with HAdV-C5 compared with HAdV-D10 (P=<0.0001) 48 hours post intravenous administration. Significantly lower levels of GFP expression were also observed in the spleen in mice administered intravenously with HAdV-D10 (FIG. 14). Taken together, these data indicate that HAdV-D10 lacks key binding residues for FX interactions and is unable to engage and utilize FX as a means of cell entry.

HAdV-D10 Vector does not Use DSG2 or CD46 for Cell Entry

We assessed the ability of HAdV-D10 vector to use DSG2 and CD46 receptors. A newly generated CHO cell line, referred to as CHO-DSG2, was developed in house. Flow cytometry analysis showed 93% of the population was positive for DSG2 expression compared to an IgG control (FIG. 3E). CHO-K1 and CHO-DSG2 cells were infected with HAdV-C5 and HAdV-D10 vectors with HAdV-C5.3 as a positive control for DSG2 binding. No significant transduction was observed in the CHO-DSG2 compared to CHO-K1, which confirms HAdV-D10 is not able to use DSG2 as a receptor for cellular entry (FIG. 3F).

A recent study suggests several species D viruses can interact with CD46 via direct engagement of the hexon. Using the whole serotype, we investigated whether Ad10 can engage CD46 as an entry receptor (FIG. 3G). There was no significant increase in infection of CHO cells expressing CD46 compared to CHO-K1 cells with both HAdV-C5 and HAdV-D10. Increased transduction was observed in CHO-CAR cells with both viral vectors (p=<0.0001 and <0.001 respectively), indicating that Ad10 does not engage CD46 as a cellular receptor.

### HAdV-D10 can be Retargeted to Av36 Integrin Through Insertion of A20 Peptide

Vectorised HAdV-D10 and the HAdV-C5/kn10 pseudotype were then evaluated as potential vectors for cancer virotherapy applications. We incorporated the A20 peptide into the previously described HAdV-C5 NULL vector to engineer selective tumour targeting (27). A20 (NAVPNLRGDLQVLAQKVART; SEQ ID NO: 1) is a 20aa long peptide from foot and mouth disease virus (FMDV) that has high selectivity and affinity for αvβ6 integrin, which is not expressed on normal epithelial cells, but commonly expressed on the surface of aggressively transformed epithelial cells, in particular malignancies of pancreatic, breast, oesophageal and ovarian origins. Incorporation of the A20 peptide into the adenovirus fiber knob has been shown to retarget the virus to these cancer cells. We inserted this peptide into the DG loop of HAdV-D10 fiber knob. SWISS-MODEL homology modelling was used to predict structure and compare HAdV-D10 fiber knob expressing the A20 peptide (FIG. 4A). This modelling was based on the fiber knob structure of HAdV-D19p as they share similar sequence identities (97.24%). Incorporation into the DG loop results in an exposed A20 peptide (green) that is able to interact with αvβ6 integrins on the surface of tumour cells. BT20 cells were used as a model cell line for evaluating the HAdV-C5/kn10.DG.A20 vector as they express high levels of αvβ6 and relatively low CAR levels (FIG. 4B). Transduction of HAdV-C5/kn10.DG.A20 was measured using a luciferase assay in BT20 cells. Cells were infected with 5000 vp/cell of HAdV-C5, HAdV-D10, HAdV-C5/kn10 or HAdV-C5/kn10.DG.A20 and luciferase output was measured at 48 hours post infection (FIG. 4C). HAdV-C5/kn10.DG.A20 produced significantly higher levels (p=<0.0001) of luciferase compared to HAdV-C5, HAdV-D10 and HAdV-C5/kn10 which had low infectivity in this this cell line. A blocking assay was performed in order to confirm this transduction data was a result of entry through the αvβ6 integrin (FIG. 4D). Experimental conditions were maintained as previously described with the exception that BT20 cells were preincubated with anti-IgG and anti-αvβ6 antibody for 30 minutes and viral infection was carried out for one hour on ice to ensure the antibody remained bound to the specific receptors. We have shown that transduction of HAdV-C5/kn10.DG.A20 in virus only conditions is significantly increased in comparison to HAdV-C5, HAdV-D10 and HAdV-C5/kn10 in accordance with the previous data (p=<0.0001). Incubation with the anti-αvβ6 antibody resulted in a significantly reduced transduction compared to virus only and IgG controls (p=<0.0001). We have observed that use of a specific anti-αvβ6 antibody to block the αvβ6 integrin prevents virus binding. These results indicate that HAdV-C5/kn10.DG.A20 can engage and utilise αvβ6 integrin as a tumour selective cell entry receptor mediated by the A20 peptide.

HAdV-D10.DG.A20 Infects Multiple Cancer Cell Lines Via αvβ6 Integrin

In addition to generating a pseudotyped αvβ6 targeted HAdV-C5/kn10.DG.A20 vector, we generated whole HAdV-D10 serotype targeting αvβ6 through insertion of A20 into HAdV-D10 vector in the same position as the HAdV-C5/kn10.DG.A20 vector. To determine the cancer selectivity of these vectors we evaluated transduction in several cancer cell lines expressing varying levels of αvβ6 integrin and CAR (FIG. 5). A549, BT20 and Kyse 30 cell lines have been derived from lung carcinoma, breast carcinoma and esophageal squamous cell carcinoma, respectively. Cell surface levels of αvβ6 integrin and CAR were assessed by flow cytometry and are indicated in the table. A549 are considered negative for αvβ6 integrin and CAR positive and Kyse 30 cells express high levels of both αvβ6 integrin and CAR.

HAdV-C5.RGE.KO1.A20 refers to an HAdV-C5 vector modified to present the A20 peptide in the HI loop of the fiber knob domain, that also possesses a RGD/E mutation in the penton base to prevent binding to cellular integrins and mutations in the fiber knob domain (KO1) to ablate CAR binding—therefore this virus is ablated for native cellular receptors and can only infect cells expressing αvβ6 integrin. As expected, αvβ6 integrin engaging viruses were unable to infect A549 cells due to lack of the αvβ6 receptor. HAdV-C5.RGE.KO1.A20 readily infects both αvβ6 expressing BT20 and Kyse 30 cell lines. Interestingly, HAdV-D10 exhibits a limited infectivity in all three cell lines. Introduction of the A20 peptide significantly increased the ability of HAdV-D10.A20 to infect both BT20 and Kyse 30 cell lines (p=<0.0001) via αvβ6 integrin.

### Increased Transduction of Labelled HAdV-D10.DG.A20 in Kyse 30 Cells

HAdV-D10 and HAdV-D10.A20 labelled with Alexa Flour 488 were used to compare intracellular trafficking of these viruses. Kyse-30 cells were seeded on a coverslip prior to infection at 25000 vp/cell and 50000 vp/cell. Cells were fixed and mounted 72 hours post infection. A confocal microscope was used to evaluate the differences in viral transduction (FIG. 6A). As previously demonstrated, there was increased uptake of HAdV-D10.A20 in the Kyse 30 cells at both concentrations of virus. This data has been quantified as number of virus particles per cell (FIG. 6B). HAdV-D10 was shown to contain significantly less virus per cell (p=<0.0001) than HAdV-D10.A20 which supports the transduction data shown in FIG. 5.

### HAdV-D10.A20 is not Neutralised in the Presence of Highly Neutralising Serum

A major obstacle contributing to reduced efficacy of HAdV-C5 based oncolytics are the proportion of patients presenting with pre-existing immunity to HAdV-C5. Use of alternative, rarely serotypes such as HAdV-D10 may provide therapies that are effective for a broader population but also offer a valuable second line of treatment in the case patients develop immunity to HAdV-C5 based virotherapies over the course of their disease. We investigated the effect of preincubation of HAdV-C5 and HAdV-D10 vectors with patient serum known to be highly neutralising against HAdV-C5 (FIG. 7A) on transduction of KYSE-30 cells.

HAdV-C5 was effectively neutralised even at the lowest concentration of serum (2.5%). HAdV-C5.RGE.KO1.A20 required higher concentrations of serum but could be effectively neutralised by the presence of >20% serum. It was not possible to detect any effect of neutralising serum in the case of HAdV-D10 due to the low level of infectivity at all concentrations. HAdV-D10.A20 however was able to infect the Kyse 30 cells and resist neutralization even at the highest concentration of serum tested (40%). When quantified as fold change (FIG. 7B), this resulted in a fluctuation of fold change between 0.7-1.5 for HAdV-D10.A20 compared to >2000-fold decrease in infection for both HAdV-C5 (6872-fold) and HAdV-C5.RGE.KO1.A20 (2100 fold) in 40% serum. To confirm these changes were statistically significant we performed an analysis comparing each serum dilution to the cell infected with virus only, which are plotted separately for clarity (FIG. 7C). HAdV-C5 and HAdV-C5.RGE.KO1.A20 demonstrated significant reduction in infection in the presence of serum at every concentration (p=<0.0001). HAdV-D10 did not show any significance due to its basal levels of infectivity. HAdV-D10.A20 did not show any change in significance indicative of decreased efficacy as a result of neutralising antibodies up to 20% serum. HAdV-D10.A20 infection was reduced compared to virus only in the highest concentration (p=0.05), but this was a small effect in comparison to that seen in the HAdV-C5 based vectors. These data suggest HAdV-D10 could provide promising vectors that circumvent the problems surrounding neutralisation observed with HAdV-C5 based vectors.

We assessed αvβ6 targeting in vivo. Female NSG mice bearing sub-cutaneous BT20 xenografts were inoculated systemically with GFP expressing HAdV vectors via intravenous injection. Liver and tumour were harvested 72 hours post infection (FIG. 7D). Variation was observed within the individual cohorts (n=5). There was no significant change in GFP levels in the liver, lung, and spleen HAdV-D10 and HAdV-D10.A20, however there was a significant increase of GFP expression mediated by HAdV-D10.A20 observed in the tumour (p=<0.05) compared to HAdV-D10.

Enhanced αvβ6 Dependent Tumour Cell Killing Using a Wild Type Replication Competent HAdV-D10.A20 Virotherapy

We investigated the tumour cell killing of wildtype replication competent HAdV-D10.A20 as a virotherapy. Two cell lines were infected with 5000 vp/cell of HAdV-C5, HAdV-D10 and HAdV-D10.A20 wild types (FIG. 8). Cell viability was measured at 72 hours post infection using CellTiter-Glo® Luminescent Cell Viability Assay (Promega). We have demonstrated that wild type HAdV-D10.A20 can infect BT20 cells via αvβ6 integrin causing significant cell death (p=<0.0001). No cell killing by HAdV-D10.A20 was observed in A549 cells that do not express αvβ6 integrin. Wild type HAdV-C5 infected A549 cells causing a loss of viability (p=<0.0001) however no significant effect was observed in the low CAR BT20 cell line. Wild-type HAdV-D10 did not cause significant cell death in either A549 or BT20 cells at 72 hours.

Based on the in vitro efficacy, we then determined whether this could be effective in vivo. Nude mice bearing BT20 xenografts were injected intratumorally with replication competent virotherapies, and the effects on tumour growth was monitored (FIG. 8B). Direct intratumoral administration of HAdV-D10.A20 resulted in significant reduction in tumour volume after eight days when compared with HAdV-D10 and PBS. No obvious signs of unexpected toxicity or weight loss were observed in mice treated with HAdV-D10 and HAdV D10.A20 virotherapies. Tumour sections were stained for gamma H2AX, a marker of cell death, which was observed in HAdV-D10.A20 treated tumours but not in HAdV-D10 or PBS treated tumours (FIG. 15).

### Biodistribution of HAdV-D10 and HAdV-D10.A20 In Vivo

Finally, we assessed the biodistribution of HAdV-D10 and HAdV-D10.A20 in vivo. Female NSG mice bearing sub-cutaneous BT-20 xenografts were inoculated with GFP expressing HAdV vectors. Liver, lung, spleen, heart, kidney, and tumours were harvested 72 hours post infection. Protein was extracted according to GFP SimpleSTEP ELISA kit (Abcam) and GFP levels measured in 50μ total protein (FIG. 9). HAdV-D10.A20 was more prevalent in each of the organs except the heart. Despite the elevation, there was not a significant change in GFP levels in the liver, lung, heart, kidney, and spleen HAdV-D10 and HAdV-D10.A20. Significant increase of HAdV-D.A20 was observed in the tumour (p=<0.05) compared to HAdV-D10.

### DISCUSSION

We have evaluated the suitability of a novel species D adenovirus, HAdV-D10 for use as virotherapy. Literature surrounding HAdV-D10 is extremely limited and mainly focus on its pathology. Therefore, we aimed to further our understanding of the receptor interactions by studying the fiber knob structure and its binding capabilities including binding with CAR, CD46 and DSG2. DSG2 binding is of particular interest, despite having the weakest interaction since this has only been described as a receptor for species BII adenoviruses previously. HAdV-B3, a well described DSG2 user, also binds DSG2 with low affinity. On further investigation, we demonstrated HAdV-D10 was not able to infect CHO-DSG2 cells and therefore consider that like other species D adenoviruses HAdV-D10 does not use DSG2 as a cellular receptor. The highest HAdV-D10 affinity interaction observed was with CAR. Using recombinant HAdV-D10k protein we confirmed that it binds CAR in vitro with a 16.5-fold lower affinity than HAdV-C5k as indicated by the IC₅₀ values. We concluded that although the binding is weak, HAdV-D10 can bind and use CAR as an entry receptor. In addition, we determined HAdV-D10 only forms weak interactions with CD46 and that it is not sufficient to infect cells. Preliminary experiments suggest HAdV-D10 may be capable of utilizing sialic acid as a mechanism for cell entry.

Alignment of HAdV-C5 and HAdV-D10 hypervariable regions identified that the key FX binding regions present in HAdV-C5 HVR7 were not present in HAdV-D10 hexon. HAdV-D10 does not interact with FX, and therefore that HAdV-D10-based virotherapies may bypass the “off target” sequestration in the liver observed using HAdV-C5-based therapies.

The A20 peptide was inserted into the DG loop of the fiber knob of HAdV-D10. A20 has previously been used to retarget HAdV-C5 to αvβ6 integrin which is upregulated in a number of cancer types including ovarian, pancreatic, breast and oesophageal cancer.

We also investigated the effect of neutralising antibodies found in patient serum on viral transduction. HAdV-D10.A20 was not neutralised even in the presence of 40% serum suggesting HAdV-D10 may provide an attractive alternative to the currently used HAdV-C5-based virotherapies to avoid pre-existing immunity.

We investigated tumour specific cell killing using wildtype and HAdV-D10.A20. HAdV-D10.A20 was able to infect BT20 cells through engagement of the αvβ6 integrin resulting in significant cell death.

We compared the tumour and liver uptake of GFP expressing vectors in vivo following systemic application and demonstrated increased tumour selective transduction through incorporation of A20 peptides, whilst transduction of other of targeted tissue was not enhanced, indicating successful targeting of HAdV-D10.A20 to αvβ6 positive tumours following systemic administration.

Finally, we investigated the tumoricidal activity of HAdV-D10.A20 in αvβ6low/CARhigh A549 and αvβ6high/CARlow BT20 cells. HAdV-C5 killed A549 cells via CAR but not BT20 cells. HAdV-D10 demonstrated consistently low levels of activity in both cell lines. HAdV-D10.A20 infected BT20 cells through engagement of αvβ6 integrin, resulting in significant cell death. We administered HAdV-D10 and HAdV-D10.A20 to mice bearing BT20 xenografts via IT injection and observed a significant decrease in tumour volume nine days post administration of HAdV-D10.A20 when compared to PBS and HAdV-D10. We have therefore developed a highly tumour selective version of HAdV-D10 that is capable of cancer specific cell killing and has shown efficacy in vivo without the need for additional de-targeting modifications.

We have therefore developed a highly targeted version of HAdV-D10 that is capable of cancer specific cell killing and has shown efficacy in vivo without the need for additional de-targeting modifications.

### SUMMARY

We have generated the first reported structure of the HAdV-D10 fiber knob and demonstrated HAdV-D10k is capable of forming weak interactions with several known adenoviral receptors including CAR, CD46, DSG2 and sialic acid. We demonstrated that HAdV-D10 does not bind FX—a feature that may improve the pharmacokinetics of HAdV-D10 based virotherapies when delivered systemically, reducing “off target” uptake in the liver. We have identified that HAdV-D10 has a limited level of infectivity across cell lines. Using this as a platform we have engineered in tumour selectivity; we successfully generated a HAdV-D10.A20 virus which infects and kills cancer cells with upregulated αvβ6 integrin expression, even in the presence of highly neutralising serum. Our findings therefore highlight that retargeted HAdV-D10 based vectors may offer significant therapeutic potential, combining reduced off target interactions with native receptors providing a platform to engineer tropism towards high affinity “on target” tumour associated receptors, with a capacity to circumvent pre-existing anti-HAdV-C5 immunity in the population. HAdV-D10 may provide therapies that are effective for a broader population but also offer a valuable second line of treatment in the case of patients who acquire treatment related immunity to HAdV-C5 based virotherapies.

Such virotherapies therefore hold significant promise as platforms for successful systemic delivery of immunovirotherapies.

TABLE 1
HAdv-D10 Fiber Knob Protein Crystal Statistics - Two Forms

| | Protein | | | |
|---|---|---|---|---|
| | Ad10k - hi | | Ad10k - lo | |
| | PDB Entry | | | |
| | 6ZC5 | | 6QPM | |
| Data Collection | | | | |
| Diamond Beamline | DLS I04-1 | | DLS I04-1 | |
| Date | 2018 Oct. 30 | | 2018 Mar. 12 | |
| Wavelength | 0.91587 | | 0.91587 | |
| Crystallisation Conditions | 0.2M CaCl2, 0.1 MES, 20% w/v | | 0.2M Na NO3, 0.1M Bis-Tris | |
| | PEG 6000 | | Propane, 20.0% PEG 3350 | |
| pH | 6.0 | | 7.5 | |
| Crystal Data | | | | |
| a, b, c (Å) | 101.357, 101.357, 326.717 | | 183.57, 183.57, 94.88 | |
| α, β, γ (°) | 90.0, 90.0, 120.0 | | 90.0, 90.0, 120.0 | |
| Space group | P3₁ | | P 6₃ | |
| Resolution (Å) | 2.50-108.91 | | 3.395-79.49 | |
| Outer shell | 2.50-2.54 | | 3.39-3.58 | |
| R-merge (%) | 14.7 | ((276.6) | 10.0 | (245.5) |
| R-pim | 6.7 | (123.1) | 3.1 | (74.7) |
| R-meas (%) | 16.2 | (303.0) | 10.4 | (256.9) |
| CC1/2 | 0.996 | (0.732) | 0.999 | (0.426) |
| I/σ(I) | 6.7 | (0.8) | 15.5 | (1.0) |
| Completeness (%) | 98.7 | (99.3) | 99.6 | (97.1) |
| Multiplicity | 5.8 | (6.0) | 11.3 | (11.3) |
| Total Measurements | 741,038 | (38,530) | 285,257 | (40,066) |
| Unique Reflections | 128,287 | (6,440) | 25,265 | (3,551) |

| | | |
|---|---|---|
| Wilson B-factor(Å²) | 57.2 | 118.1 |
| Refinement Statistics | | |
| Non-H Atoms | 17,536 | 8,694 |
| R-work reflections | 121,851 | 23,861 |
| R-free reflections | 6,341 | 1,235 |
| R-work/R-free (%) | 21.9/25.1 | 20.3/23.4 |
| ¹Twin Law 1/Fraction 1 | H, K, L/0.253 | n/a |
| ¹Twin Law 2/Fraction 2 | K, H, −L/0.254 | n/a |
| ¹Twin Law 3/Fraction 3 | −H, −K, L/0.245 | n/a |
| ¹Twin Law 4/Fraction 4 | −k, −H, −L/0.248 | n/a |
| ²rms deviations | | |
| Bond lengths (Å) | 0.013 | 0.008 |
| Bond Angles (°) | 1.576 | 1.753 |
| ³Coordinate error | 0.050 | 0.480 |
| Mean B value (Å²) | 78.7 | 172.4 |
| Ramachandran Statistics | | |
| Favoured/allowed/Outliers | 1886/271/30 | 982/99/5 |
| % | 86.2/12.4/1.4 | 90.4/9.1/0.5 |

* One crystal was used for determining the structure.
* Figures in brackets refer to outer resolution shell, where applicable.
¹Twin laws determined and fractions estimated automatically by REFMAC5
²Figures in brackets are rms targets
³Coordinate Estimated Standard Uncertainty in (Å), calculated based on maximum likelihood statistics.

TABLE 2
Primers used in generation of viral vectors

| Primer | Sequence (5′-3′) |
|---|---|
| Cass in E4Orf6 Ad10 | CGGTGATTGAGATGAAGCCGTCCTCTGAAAAGTCATCCAAGCGAGCCTCA |
| F | CAGTCCAAGGCCTGTGACGGAAGATCACTTCG (SEQ ID NO: 3) |
| Cass in E4Orf6 Ad10 | GTTCAAGGGCCCATTTCTGCTGGCAGAAGTACGACAAGGTACGCAAGAGA |
| R | ATCCACTACACTGAGGTTCTTATGGCTCTTG (SEQ ID NO: 4) |
| Ad5 E4Orf6 Ad10 | CGGTGATTGAGATGAAGCCGTCCTCTGAAAAGTCATCCAAGCGAGCCTCA |
| arms F | CAGTCCAAGGCTACATGGGGGTAGAGTCATAATCG (SEQ ID NO: 5) |
| Ad5 E4Orf6 Ad10 | GTTCAAGGGCCCATTTCTGCTGGCAGAAGTACGACAAGGTACGCAAGAGA |
| arms R | ATCCACTACAATGACTACGTCCGGCGTTCCATTTG (SEQ ID NO: 6) |
| Cass in E3 Ad10 F | TGGTCAGGTTCTTCACCCAGCAACCCTTCCTGGTCGAGCGGGACCGGGGC |
| | GCCACCACCTACACCGTCTACCTGTGACGGAAGATCACTTCG (SEQ ID |
| | NO: 7) |
| Cass in E3 Ad10 R | GGTTTGATTGGTTTCTGGGCTTTAATCAACATCAGTTCATGGGCAGGAGG |
| | TCGCGGAGTCCGCAAAGGGTCTGAGGTTCTTATGGCTCTTG (SEQ ID |
| | NO: 8) |
| Ad10 E3 delete | CAACCCTTCCTGGTCGAGCGGGACCGGGGCGCCACCACCTACACCGTCTA |
| oligo | ACCCTTTGCGGACTCCGCGACCTCCTGCCCATGAACTGATGTTGATTAAA |
| | (SEQ ID NO: 9) |
| CMV/polyA in E1 F | GTCAAGAGGCCACTCTTGAGTGCCAGCGAGTAGAGATTTCTCTGAGCTCC |
| | GCTCCCAGAGACCGAGAAAACCTGTGACGGAAGATCACTTCG (SEQ ID |
| | NO: 10) |
| CMV/polyA in E1 R | AAAAAGACCCTCGTAAGACACCCGCCTTTATAGTCACCTTAGCCACGCCC |
| | ACTACTCACTCGACCTACCTCTGAGGTTCTTATGGCTCTTG (SEQ ID |
| | NO: 11) |
| RPSL/sacB kn10.DG F | TTATGAAAAAGCAATTGGTTTTATGCCTAATTTGGTAGCGTATCCGAAAC |
| | CCAGTAATTCTAAAAAATATCCTGTGACGGAAGATCACTTCG (SEQ ID |
| | NO: 12) |
| RPSL/sacB kn10.DG R | TAGTTTTAATGACTGCTGGCTGATCAGGTTTTCCACCAAGATATATAGTT |
| | CCATAAACTATGTCTCTTGCCTGAGGTTCTTATGGCTCTTG (SEQ ID |
| | NO: 13) |
| kn10.DG.A20 F | TTATGAAAAAGCAATTGGTTTTATGCCTAATTTGGTAGCGTATCCGAAAC |
| | CCAGTAATTCTAAAAAATATAATGCTGTGCCCAACTTGAGAGGTGAC |
| | (SEQ ID NO: 14) |
| kn10.DG.A20 R | TAGTTTTAATGACTGCTGGCTGATCAGGTTTTCCACCAAGATATATAGTT |
| | CCATAAACTATGTCTCTTGCCGTCCGTGCCACCTTTTGAGCCAAC (SEQ |
| | TD NO: 15) |
| PCR HAdV-D10 knob F | AACACCAGACACTTCTCCAAACTGCAC (SEQ ID NO: 16) |
| PCR HAdV-D10 knob R | TACACTGTGAAATGGGCTGGTGGTGG (SEQ ID NO: 17) |
| Seq HAdV-D10 knob F | ATTGCTCAGGATAAGGACTCTAAACTAACTC (SEQ ID NO: 18) |
| Seq HAdV-D10 knob R | AGACTGACTACCCGTGCTGGTGTAAAAATC (SEQ ID NO: 19) |

### REFERENCES

27. H. Uusi-Kerttula, et al., Ad5NULL-A20—a tropism-modified, αvβ6 integrin-selective oncolytic adenovirus for epithelial ovarian cancer therapies. 24, 4215-4224 (2018).
48. A. T. Baker, et al., Diversity within the adenovirus fiber knob hypervariable loops influences primary receptor interactions. Nature Communications 10, 741 (2019).
50. R. Alba, et al., Identification of coagulation factor (F)X binding sites on the adenovirus serotype 5 hexon: effect of mutagenesis on FX interactions and gene transfer. Blood 114, 965-971 (2009).
33. H. Uusi-Kerttula, et al., Pseudotyped αvβ6 integrin-targeted adenovirus vectors for ovarian cancer therapies. Oncotarget 7, 27926-27937 (2016).
49. R. J. Stanton, B. P. McSharry, M. Armstrong, P. Tomasec, G. W. G. Wilkinson, Re-engineering adenovirus vector systems to enable high-throughput analyses of gene function. BioTechniques 45, 659-668 (2008).

1. A modified D species human adenovirus of serotype 10, HAdV-D10, comprising:
a binding epitope with selective affinity for αvβ6 integrin, comprising or consisting of a 20 amino acid peptide, NAVPNLRGDLQVLAQKVART (SEQ ID NO: 1), termed A20 and native to foot and mouth disease virus (termed HAdV-D10.A20); and any one or more of the following features:
a) an IC₅₀ for the coxsackievirus and adenovirus receptor (CAR) greater than 0.001 μg/10⁵ cells; and/or
b) a lack of binding to human coagulation factor X (FX); and/or
c) transduction ability in the presence of serum neutralising anti-HAdV-C antibodies.

2. The modified adenovirus according to claim 1 wherein said A20 peptide is inserted into the DG loop of HAdV-D10 fiber knob protein.

3. The modified adenovirus according to claim 1 wherein said IC₅₀ for the coxsackievirus and adenovirus receptor (CAR) is greater than 0.002 μg/10⁵ cells, or about 0.003 μg/10⁵ cells.

4. The modified adenovirus according to claim 1 wherein HAdV-D10 binds CAR with an apparent 10-fold lower affinity or a 15-fold lower affinity, or 16.5-fold lower affinity than HAdV-C5.

5. The modified adenovirus according to claim 1 wherein HAdV-D10.A20, lacks key binding residues for FX interactions and so is unable to engage FX.

6. The modified adenovirus according to claim 1 wherein said HAdV-D10.A20 is not affected by the presence of pre-existing immunity to other adenoviruses.

7. The modified adenovirus according to claim 1 wherein a molecule encoding at least one transgene is inserted into said adenovirus.

8. The modified adenovirus according to claim 7 wherein said molecule is cDNA encoding at least one or each transgene.

9. The modified adenovirus according to claim 1 wherein said adenovirus is further modified to include a 24-base pair deletion d1922-947 (Δ24 mutation) in the ELA gene to restrict viral replication to pRB-defective cells.

10. The modified adenovirus according to claim 1 said adenovirus is further modified to include a single adenine base addition at position 445 within the endoplasmic reticulum (ER) retention domain in E3/19K (T1 mutation) for enhanced oncolytic potency.

11. The modified adenovirus according to claim 1 said adenovirus is further modified to include an adenovirus death protein (ADP).

12. The modified adenovirus according to claim 11 wherein the ADP is from an adenovirus selected from the group comprising: Ad1, Ad2, Ad5, Ad6, and Ad57.

13. The modified adenovirus according claim 1 wherein said adenovirus is further modified wherein the E4orf6 region was replaced with the HAdV-C5 E4orf6 to improve viral propagation.

14. A pharmaceutical composition comprising the modified adenovirus according claim 1 and a pharmaceutically acceptable carrier, adjuvant, diluent, or excipient.

15. A method of treating cancer in a subject comprising administering to a patient an effective amount of the modified adenovirus according to claim 1.

16. The modified adenovirus according to claim 1 for use as a medicament.

17. The modified adenovirus according to claim 1 for use in the treatment of cancer.

18. A method of using the modified adenovirus of claim 1 in the manufacture of a medicament to treat cancer.

19. The method according to claim 15 wherein the cancer is selected from the group comprising: nasopharyngeal cancer, synovial cancer, hepatocellular cancer, renal cancer, cancer of connective tissues, melanoma, lung cancer, bowel cancer, colon cancer, rectal cancer, colorectal cancer, brain cancer, throat cancer, oral cancer, liver cancer, bone cancer, pancreatic cancer, choriocarcinoma, gastrinoma, pheochromocytoma, prolactinoma, T-cell leukemia/lymphoma, neuroma, von Hippel-Lindau disease, Zollinger-Ellison syndrome, adrenal cancer, anal cancer, bile duct cancer, bladder cancer, ureter cancer, brain cancer, oligodendroglioma, neuroblastoma, meningioma, spinal cord tumor, bone cancer, osteochondroma, chondrosarcoma, Ewing's sarcoma, cancer of unknown primary site, carcinoid, carcinoid of gastrointestinal tract, fibrosarcoma, breast cancer, Paget's disease, cervical cancer, colorectal cancer, rectal cancer, esophagus cancer, gall bladder cancer, head cancer, eye cancer, neck cancer, kidney cancer, Wilms' tumor, liver cancer, Kaposi's sarcoma, prostate cancer, lung cancer, testicular cancer, Hodgkin's disease, non-Hodgkin's lymphoma, oral cancer, skin cancer, mesothelioma, multiple myeloma, ovarian cancer, endocrine pancreatic cancer, glucagonoma, pancreatic cancer, parathyroid cancer, penis cancer, pituitary cancer, soft tissue sarcoma, retinoblastoma, small intestine cancer, stomach cancer, thymus cancer, thyroid cancer, trophoblastic cancer, hydatidiform mole, uterine cancer, endometrial cancer, vagina cancer, vulva cancer, acoustic neuroma, mycosis fungoides, insulinoma, carcinoid syndrome, somatostatinoma, gum cancer, heart cancer, lip cancer, meninges cancer, mouth cancer, nerve cancer, palate cancer, parotid gland cancer, peritoneum cancer, pharynx cancer, pleural cancer, salivary gland cancer, tongue cancer and tonsil cancer.

20. The method according to claim 15 wherein said cancer is selected from the group comprising: ovarian cancer, pancreatic cancer, oesophageal cancer, lung cancer, cervical cancer, head and neck cancer, oral cancer, cancer of the larynx, skin cancer, breast cancer, kidney cancer, and colorectal cancer.

21. A modified adenovirus according to claim 1 for use in the treatment of cancer in a subject wherein said subject exhibits pre-existing immunity to adenovirus

22. The modified adenovirus according to claim 21 wherein said subject exhibits pre-existing immunity to any one or more of: HAdV-C5, HAdV-E4, HAdV-B11, HAdV-D26, HAdV-48, the chimera HAdV-5/3 and Chimp adenovirus.


SEQUENCE LISTING (19 sequences)

SEQ ID NO: 1 - protein, 20 aa, Foot-and-mouth disease virus
NAVPNLRGDLQVLAQKVART

SEQ ID NO: 2 - protein, 8 aa, Unknown
  Adenovirus type 10 E1A gene mutation
LRCYEEGF

SEQ ID NO: 3 - DNA, 82 bp, Artificial Sequence
  Cass in E4Orf6 Ad10 F primer
cggtgattgagatgaagccgtcctctgaaaagtcatccaagcgagcctcacagtccaaggcctgtgacggaagatcacttcg

SEQ ID NO: 4 - DNA, 81 bp, Artificial Sequence
  Cass in E4Orf6 Ad10 R primer
gttcaagggcccatttctgctggcagaagtacgacaaggtacgcaagagaatccactacactgaggttcttatggctcttg

SEQ ID NO: 5 - DNA, 85 bp, Artificial Sequence
  Ad5 E4Orf6 Ad10 arms F primer
cggtgattgagatgaagccgtcctctgaaaagtcatccaagcgagcctcacagtccaaggctacatgggggtagagtcataatcg

SEQ ID NO: 6 - DNA, 85 bp, Artificial Sequence
  Ad5 E4Orf6 Ad10 arms R primer
gttcaagggcccatttctgctggcagaagtacgacaaggtacgcaagagaatccactacaatgactacgtccggcgttccatttg

SEQ ID NO: 7 - DNA, 92 bp, Artificial Sequence
  Cass in E3 Ad10 F primer
tggtcaggttcttcacccagcaacccttcctggtcgagcgggaccggggcgccaccacctacaccgtctacctgtgacggaagatcacttcg

SEQ ID NO: 8 - DNA, 91 bp, Artificial Sequence
  Cass in E3 Ad10 R primer
ggtttgattggtttctgggctttaatcaacatcagttcatgggcaggaggtcgcggagtccgcaaagggtctgaggttcttatggctcttg

SEQ ID NO: 9 - DNA, 100 bp, Artificial Sequence
  Ad10 E3 delete oligo primer
caacccttcctggtcgagcgggaccggggcgccaccacctacaccgtctaaccctttgcggactccgcgacctcctgcccatgaactgatgttgattaaa

SEQ ID NO: 10 - DNA, 92 bp, Artificial Sequence
  CMV/polyA in E1 F primer
gtcaagaggccactcttgagtgccagcgagtagagatttctctgagctccgctcccagagaccgagaaaacctgtgacggaagatcacttcg

SEQ ID NO: 11 - DNA, 91 bp, Artificial Sequence
  CMV/polyA in E1 R primer
aaaaagaccctcgtaagacacccgcctttatagtcaccttagccacgcccactactcactcgacctacctctgaggttcttatggctcttg

SEQ ID NO: 12 - DNA, 92 bp, Artificial Sequence
  RPSL/sacB kn10.DG F primer
ttatgaaaaagcaattggttttatgcctaatttggtagcgtatccgaaacccagtaattctaaaaaatatcctgtgacggaagatcacttcg

SEQ ID NO: 13 - DNA, 182 bp, Artificial Sequence
  RPSL/sacB kn10.DG R primer
tagttttaatgactgctggctgatcaggttttccaccaagatatatagttccataaactatgtctcttgcctgaggttcttatggctcttgtagttttaatgactgctggctgatcaggttttccaccaagatatatagttccataaactatgtctcttgcctgaggttcttatggctcttg

SEQ ID NO: 14 - DNA, 97 bp, Artificial Sequence
  kn10.DG.A20 F primer
ttatgaaaaagcaattggttttatgcctaatttggtagcgtatccgaaacccagtaattctaaaaaatataatgctgtgcccaacttgagaggtgac

SEQ ID NO: 15 - DNA, 95 bp, Artificial Sequence
  kn10.DG.A20 R primer
tagttttaatgactgctggctgatcaggttttccaccaagatatatagttccataaactatgtctcttgccgtccgtgccaccttttgagccaac

SEQ ID NO: 16 - DNA, 27 bp, Artificial Sequence
  PCR HAdV-D10 knob F primer
aacaccagacacttctccaaactgcac

SEQ ID NO: 17 - DNA, 26 bp, Artificial Sequence
  PCR HAdV-D10 knob R primer
tacactgtgaaatgggctggtggtgg

SEQ ID NO: 18 - DNA, 31 bp, Artificial Sequence
  Seq HAdV-D10 knob F
attgctcaggataaggactctaaactaactc

SEQ ID NO: 19 - DNA, 30 bp, Artificial Sequence
  Seq HAdV-D10 knob R primer
agactgactacccgtgctggtgtaaaaatc